(19) United States
(12) Patent Application Publication
(10) Pub. No.: US 2026/0284105 A1
(43) Pub. Date: Sep. 24, 2026

(54) METHODS OF PRODUCING MATURED DENDRITIC CELLS FOR IMMUNOTHERAPY

(71) Applicants: The Education University of Hong Kong, New Territories (CN); The Chinese University of Hong Kong, New Territories (CN); Hong Kong Baptist University, Kowloon Tong, Hong Kong (CN)

(72) Inventors: Kin Lam Yung, New Territories (CN); Zhifeng Huang, New Territories (CN); Shiqing Zhang, Kowloon Tong (CN)

(21) Appl. No.: 19/569,056

(22) Filed: Mar. 17, 2026

Related U.S. Application Data
(60) Provisional application No. 63/775,439, filed on Mar. 21, 2025.

(51) Int. Cl. A61K 35/15; A61K 9/00; A61K 40/19; A61K 40/42; A61P 35/00; C12N 5/0784

(52) U.S. Cl. CPC A61K 35/15; A61K 9/0019; A61K 40/19; A61K 40/42; A61P 35/00; C12N 5/0639; C12N 2506/11; C12N 2533/14; C12N 2535/00

(57) ABSTRACT

Disclosed herein are methods for producing matured dendritic cells for immunotherapy. The disclosed methods include a) providing a biocompatible nanomatrix; b) seeding immature dendritic cells onto the biocompatible nanomatrix; and c) culturing the dendritic cells for a period sufficient to induce maturation via mechanical stimuli provided by the biocompatible nanomatrix. Also disclosed herein are the matured dendritic cells produced by the methods, as well as pharmaceutical compositions including the matured dendritic cells.

### TECHNICAL FIELD

The present invention relates to the field of immunology and biotechnology, and more particularly to methods for producing matured dendritic cells (DCs) suitable for use in immunotherapy. Specifically, although not exclusively, the invention provides methods of producing the matured DCs for cancer treatment.

### BACKGROUND

Treating cancer today remains a major challenge because conventional therapies-such as surgery, chemotherapy, and radiation-often come with severe side effects, limited effectiveness in advanced stages, and high rates of recurrence. Many tumors also develop resistance, leaving patients with few options.

Dendritic cell (DC) cancer vaccines offer an alternative solution by harnessing the body's own immune system to recognize and attack cancer cells. To prepare the DC cancer vaccines, the immature DCs are in vitro differentiated from the monocytes or hematopoietic stem cells isolated from the patient's blood. Afterward, the immature DCs are stimulated to adopt the maturation phenotypes and pulsed with tumor antigens, before being administered back to the patient's body to initiate the adaptive immune response against cancer cells. To maximize the efficacy of the DC vaccine, the in vitro maturated DCs shall have the optimized ability to migrate to the secondary lymphoid organs, produce cytokines, and induce the effector functions and memory formations of antigen-specific cytotoxic T-lymphocytes (CTLs).

The maturation phenotypes of DCs can be manipulated by applying different factors that typically involve cytokines and toll-like receptor (TLR) agonists. However, these maturation agents often have limitations. For example, the maturation cocktail consisting of tumor necrosis factor-alpha (TNFα), interleukin-1β, interleukin-6, and prostaglandin E2 resulted in a high yield of maturated DCs with enhanced migratory capacity but suppressed production of interleukin-12 (IL12) which is an important cytokine for anti-tumor CTL function. The TLR ligands enhanced the expression of co-stimulatory molecules and cytokines and promoted cross-presentation in DCs. However, some of the TLR ligands such as lipopolysaccharide (LPS) and polyinosinic-polycytidylic acid (poly I:C) are potentially toxic to animals. The TLR-maturated DCs would have a G-protein expression profile associated with limited migratory capacity.

Recently, there were reports discussing the possibility of using biomaterials to deliver biophysical signals for manipulating the properties of in vitro maturated DCs, including making modifications of cell culture substrates with poly(lactic-co-glycolic acid) or inorganic topographies (such as titanium disks). However, these biomaterials would either cause apoptosis or fail to increase the production of proinflammatory cytokines. As a result, the CTLs could not be efficiently activated to an extent comparable to the conventional methods.

Given the limitations and complexities of the current strategies, there remains a need to provide an alternative method for maturation of DCs.

### SUMMARY OF THE INVENTION

It would be beneficial to have an alternative method for in vitro maturation of DCs. Preferably, the method can activate CTLs in a simple and biocompatible manner.

In accordance with a first aspect of the invention, there is provided a method of producing matured dendritic cells for immunotherapy. The method comprises:
a) providing a biocompatible nanomatrix;
b) seeding immature dendritic cells onto the biocompatible nanomatrix; and
c) culturing the dendritic cells for a period sufficient to induce maturation via mechanical stimuli provided by the biocompatible nanomatrix.

In embodiments, the biocompatible nanomatrix comprises a substrate, and a plurality of nanostructures protruding from the substrate. In an embodiment, the nanostructures are composed of silica.

In embodiments, the nanostructures are configured in a zigzag geometry. Preferably, the zigzag geometry is configured to induce mechanical maturation of dendritic cells via activation of focal adhesion kinase.

In embodiments, the nanostructures comprises between two and five pitches. In some instances, each of the nanostructures comprises 3 pitches.

In embodiments, each of the pitches has a height of approximately 100 nm to 500 nm.

In embodiments, the nanostructures are fabricated via glancing angle deposition.

In embodiments, the immature dendritic cells are bone marrow-derived dendritic cells, monocyte-derived dendritic cells, or CD34⁺ hematopoietic stem cells.

In embodiments, the period sufficient to induce maturation is approximately 20 hours.

In embodiments, the maturation of step c) is performed in the absence of exogenous chemical maturation agents or cytokines.

In embodiments, the matured dendritic cells are configured to activate antigen-specific cytotoxic T-lymphocytes. Preferably, the activated antigen-specific cytotoxic T-lymphocytes exhibit a PD-1^{low}CD44^{high} memory phenotype.

In embodiments, the method further comprises pulsing the dendritic cells with at least one tumor-associated antigen.

In embodiments, the matured dendritic cells exhibit upregulated expression of a co-stimulatory and immunoregulatory molecule selected from the group consisting of CD86, DC-SIGN, XCR1, CCR7, IL-12, TNFα, and a combination thereof.

In accordance with a second aspect of the invention, there is provided matured dendritic cells prepared according to the method disclosed herein.

In accordance with a third aspect of the invention, there is provided a pharmaceutical composition comprising an effective amount of the matured dendritic cells disclosed herein.

In embodiments, the matured dendritic cells present at least one tumor-associated antigen on the MHC-I complexes. In embodiments, the matured dendritic cells exhibit upregulated expression of a co-stimulatory and immunoregulatory molecule selected from the group consisting of CD86, DC-SIGN, XCR1, CCR7, IL-12, TNFα, and a combination thereof.

The invention provides a method for producing maturated DCs by a biocompatible nanomatrix. The DCs are matured into a functional phenotype that will promote the priming of antigen-specific CTLs against tumors. The inventors have discovered that an embodiment of the biocompatible nanomatrix having silica nanozigzags (NZs), particularly those fabricated with a pitch of approximately 245 nm and three pitches, optimally promote in vitro maturation of bone marrow-derived dendritic cells (mBMDCs) through focal adhesion kinase (FAK)-mediated mechanoactivation. Functionally, when an antigen is provided, the matured mBMDCs can effectively activate antigen-specific CTLs and induce the formation of CD44^{high} effector memory CTLs without a significant increase in programmed cell death protein-1 (PD-1). Also, administration of antigen-pulsed matured mBMDCs was found to be effective in suppressing tumor growth and in improving survival in mice bearing antigen-expressing B16 melanoma. Furthermore, similar maturation-enhancing effects were observed in human monocyte-derived dendritic cells (hu-moDCs), indicating that the methods as disclosed herein can be broadly applied to activate diverse dendritic cell populations.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments of the present invention will now be described, by way of example, with reference to the accompanying drawings.

FIG. 1A is a schematic diagram of a biocompatible nanomatrix according to an embodiment of the present invention. FIG. 1B is a schematic diagram of a nanostructure configured on the biocompatible nanomatrix according to the embodiment. FIG. 1C is a schematic illustration of the effects of a biocompatible nanomatrix containing silica nanozigzags (NZs) on enhancing the in vitro maturation of anti-tumor DCs via the activation of focal adhesion kinase (FAK).

FIG. 2A to 2K show an embodiment of a method of preparing matured DCs from murine bone marrow-derived DCs (mBMDCs), and demonstrate the NZ nanostructures of the biocompatible nanomatrix can modulate the maturation of mBMDCs. FIG. 2A is a schematic of experimental timeline to prepare matured DCs from mBMDCs with different nanomatrices, and to characterize the properties of the maturated mBMDCs; in which the mBMDCs attached to the tissue culture polystyrene (TCPS), without any maturation treatment, served as the negative control group. FIG. 2B is a schematic illustration of two embodiments of the nanostructure of the biocompatible nanomatrix, including a three-pitch silica nanostructure having a pitch length of 165 nm for forming a nanomatrix NZs-P165-N3, and a three-pitch silica nanostructure having a pitch length of 245 nm for forming a nanomatrix NZs-P245-N3. FIG. 2C shows representative histograms of CD86, DC-SIGN, and XCR1 in the CD11c⁺ MHC-II⁺ mBMDCs maturated with the different nanomatrices NZs-P165-N3 and NZs-P245-N3, and the negative control group TCPS; with the number of independent trials (n) of 4. FIG. 2D is a plot of the geometric mean fluorescence intensity (MFI) of CD86 in the CD11c⁺ MHC-II⁺ mBMDCs maturated with the different nanomatrices NZs-P165-N3 and NZs-P245-N3, and the negative control group TCPS; n=4. FIG. 2E is a plot of the geometric MFI of DC-SIGN in the CD11c⁺ MHC-II⁺ mBMDCs maturated with the different nanomatrices NZs-P165-N3 and NZs-P245-N3, and the negative control group TCPS; n=4. FIG. 2F is a plot of the geometric MFI of XCR1 in the CD11c⁺ MHC-II⁺ mBMDCs maturated with the different nanomatrices NZs-P165-N3 and NZs-P245-N3, and the negative control group TCPS; n=4. FIG. 2G is a schematic illustration of silica NZs composed of P of 245 nm and the controllable number of pitch (N of 2, 3, 4, and 5). FIG. 2H shows representative histograms of CD86, DC-SIGN, and XCR1 in the CD11c⁺ MHC-II⁺ mBMDCs maturated with the nanomatrices having different silica NZs with P being 245 nm and N being any from 2 to 5 (n=4); the lines on histograms indicate the peaks in histograms of TCPS-attached mBMDCs. FIG. 2I is a plot of the geometric MFI of CD86 in the CD11c⁺ MHC-II⁺ mBMDCs maturated with the nanomatrices having different silica NZs with P being 245 nm and N being any from 2 to 5 (n=4); the error bars represent the standard error of the mean. *p<0.05, **p<0.01, ***p<0.001, ****p<0.0001, one-way ANOVA with Tukey's HSD post-hoc test. FIG. 2J is a plot of the geometric MFI of DC-SIGN in the CD11c⁺ MHC-II⁺ mBMDCs maturated with the nanomatrices having different silica NZs with P being 245 nm and N being any from 2 to 5 (n=4); the error bars represent the standard error of the mean. *p<0.05, **p<0.01, ***p<0.001, ****p<0.0001, one-way ANOVA with Tukey's HSD post-hoc test. FIG. 2K is a plot of the geometric MFI of XCR1 in the CD11c⁺ MHC-II⁺ mBMDCs maturated with the nanomatrices having different silica NZs with P being 245 nm and N being any from 2 to 5 (n=4); the error bars represent the standard error of the mean. *p<0.05, **p<0.01, ***p<0.001, ****p<0.0001, one-way ANOVA with Tukey's HSD post-hoc test.

FIG. 3A to 3L demonstrate that silica NZs-P245-N3 promotes the maturation of XCR1⁺DC⁻SIGN⁺ mBMDCs, with an enhancement of endocytic capacity and production of pro-inflammatory cytokines. FIG. 3A is a scanning electron microscopy (SEM) cross-sectional image of the silica NZs (with a P of 245 nm and three pitches, i.e., NZs-P245-N3) perpendicularly protruding on a supporting substrate; insets: (upper left) SEM image of an individual NZ (scale bar: 250 nm); (upper right) SEM top-down image of the NZs. FIG. 3B is a schematic of experimental timeline to characterize the properties of mBMDCs maturated with the silica NZs or LPS; in which the mBMDCs attached to the TCPS, without any maturation treatment, served as the negative control group. FIG. 3C shows representative histograms of CD86⁺, CCR7⁺, XCR1⁺, DC-SIGN⁺, and CD206⁺ cells in the CD11c⁺ MHC-II⁺ mBMDC subset after the 20 h maturation period. FIG. 3D is a plot showing the percentage of CD86+ cells in the CD11c⁺ MHC-II⁺ mBMDC subset after the 20 h maturation period (n=6). FIG. 3E is a plot showing the percentage of CCR7+ cells in the CD11c⁺ MHC-II⁺ mBMDC subset after the 20 h maturation period (n=4). FIG. 3F is a plot showing the percentage of XCR1⁺ cells in the CD11c⁺MHC-II⁺ mBMDC subset after the 20 h maturation period (n=4). FIG. 3G is a plot showing the percentage of DC-SIGN⁺ cells in the CD11c⁺ MHC-II⁺ mBMDC subset after the 20 h maturation period (n=4).

FIG. 3H is a plot showing the percentage of CD206⁺ cells in the CD11c⁺ MHC-II⁺ mBMDC subset after the 20 h maturation period (n=3). FIG. 3I shows representative confocal images (with yellow lines indicating the outlines of cells defined by the staining of F-actin; scale bars: I, 20 μm; K, 10 μm) demonstrating the presence of intracellular dextran in the maturated mBMDCs after a 15 min treatment of AF647-conjugated dextran. FIG. 3J is a plot showing the quantifications of fluorescence levels of intracellular dextran in the maturated mBMDCs after a 15 min treatment of AF647-conjugated dextran or dark-red fluorescent microspheres; in which at least 200 cells were measured in each group per trial, and at least 1300 cells were measured in each group for five independent trials (n=5). FIG. 3K shows representative confocal images (with yellow lines indicating the outlines of cells defined by the staining of F-actin; scale bars: I, 20 μm; K, 10 μm) demonstrating the presence of dark-red microsphere in the maturated mBMDCs after a 15 min treatment of dark-red fluorescent microspheres. FIG. 3L is a plot showing the levels of the uptake of the microspheres in the maturated mBMDCs after a 15 min treatment of dark-red fluorescent microspheres; in which at least 200 cells were measured in each group per trial, and at least 1300 cells were measured in each group for five independent trials (n=5). The error bars represent the standard error of the mean. *p<0.05, **p<0.01, ***p<0.001, ****p<0.0001, (for FIGS. 3D, 3E, 3G, 3H, 3J, and 3L) one-way ANOVA with Tukey's HSD post-hoc test, (F) Welch's ANOVA with Dunnett's T3 post-hoc test.

FIGS. 4A to 4L show characterization of cell adhesions at the DC-NZ interfaces, with an activation of FAK, in accordance with an embodiment of the present invention; in which mBMDCs were seeded on a biocompatible matrix having silica NZs-P245-N3 for 20 h, and fixed and visualized using SEM and confocal fluorescence microscopy. FIG. 4A is a representative SEM image showing an attachment of mBMDCs on the silica NZs. FIG. 4B is an enlarged view of the area indicated with the black box in FIG. 4A, where the cell membrane is shaded in a pale blue color; the white arrows show the locations where the silica NZs are in contact with the basal plasma membrane of mBMDC; the red dotted lines indicate the outline of a silica NZ. FIG. 4C is a representative maximum-intensity projection (MIP) of deconvoluted confocal z-stack showing the CellTracker Deep Red dye-stained silica NZs (red) and wheat germ agglutinin-stained plasma membrane (green) of NZ-maturated mBMDCs. FIG. 4D shows orthogonal views in the XZ plane labeled with the dotted line in FIG. 4C. FIG. 4E shows representative MIPs of deconvoluted confocal z-stacks showing the CellTracker Deep Red dye-stained silica NZs (green), immunostaining of vinculin (magenta), and phalloidin-stained F-actin (blue) in the basal plasma membrane of NZ-maturated mBMDCs; in which the inset is a schematic illustrating the formation of cell adhesions (magenta) at the interfaces between the DC (blue) and silica NZs (green). FIG. 4F is a plot showing fluorescence intensity profiles of the silica NZs, vinculin, and F-actin across the white arrow labeled on the enlarged merge view in FIG. 4E. FIG. 4G demonstrates individual slices in the z-stack of an enlarged view indicated by the orange box in FIG. 4E; in which the white arrowheads mark the depth of a vertical cell adhesion formed at the DC-NZ interfaces. FIG. 4H shows representative confocal images showing the immunostaining results of vinculin (magenta) and p-FAK (Y397, green) at the basal plasma membranes of mBMDCs attached to TCPS and silica NZs; in which the yellow arrows and arrowheads indicate the podosomes and focal adhesions, respectively, in the TCPS-attached mBMDCs. FIG. 4I is a plot of the cell spreading area covered with vinculin⁺ cell adhesions in the mBMDCs attached to TCPS or silica NZs. FIG. 4J is a plot of the percentage of cell area covered with vinculin⁺ cell adhesions in the mBMDCs attached to TCPS or silica NZs. FIG. 4K shows two plots of fluorescence intensity profiles of vinculin and p-FAK across the white arrows in FIG. 4H. FIG. 4L is a plot showing fluorescence intensity of p-FAK in the vinculin⁺ cell adhesions of mBMDCs attached to TCPS or silica NZs. The error bars represent the standard error of the mean. ****p<0.0001, two-tailed Welch's t-test, n=305 cells for the TCPS group and n=313 cells for the NZ group, pooled from five independent experimental trials.

FIGS. 5A to 5L demonstrate that silica NZs promote the maturation of mBMDCs via the activation of FAK. FIG. 5A is a schematic of the experimental timeline to examine the role of FAK in the NZs(-P245-N3)-induced maturation of mBMDCs via the application of FAK inhibitor PF-573228 (PF); in which the mBMDCs attached to the TCPS, with the treatment of 0.02% DMSO, served as the vehicle control group. FIG. 5B are representative Western blots showing the level of Tyr-397 (Y397)-phosphorylated FAK in the mBMDCs after the 20 h maturation period (n=5). FIG. 5C is a plot showing the ratio of p-FAK: total FAK in the mBMDCs after the 20 h maturation period (n=5). FIG. 5D shows representative histograms of CD86⁺, CCR7⁺, XCR1⁺, and DC-SIGN⁺ cells in the CD11c⁺ MHC-II⁺ mBMDC subset after the 20 h maturation period (n=4). FIG. 5E is a plot of the percentage of CD86+ cells in the CD11c⁺ MHC-II⁺ mBMDC subset after the 20 h maturation period (n=4). FIG. 5F is a plot of the percentage of CCR7⁺ cells in the CD11c⁺ MHC-II⁺ mBMDC subset after the 20 h maturation period (n=4). FIG. 5G is a plot of the percentage of XCR1+ cells in the CD11c⁺ MHC-II⁺ mBMDC subset after the 20 h maturation period (n=4).

FIG. 5H is a plot of the percentage of DC-SIGN⁺ cells in the CD11c⁺ MHC-II⁺ mBMDC subset after the 20 h maturation period (n=4). FIG. 5I shows representative confocal images (with yellow lines indicating the outlines of cells defined by the staining of F-actin; scale bars: 10 μm). FIG. 5J is a plot of fluorescence level of intracellular dextran in the maturated mBMDCs after a 15 min treatment of AF647-conjugated dextran; in which at least 200 cells were measured in each group per trial, and at least 1000 cells were measured in each group for five independent trials (n=5). The error bars represent the standard error of the mean. *p<0.05, **p<0.01, ***p<0.001, ****p<0.0001, one-way ANOVA with Tukey's HSD post-hoc test. FIG. 5K shows representative confocal images (with yellow lines indicating the outlines of cells defined by the staining of F-actin; scale bars: 10 μm). FIG. 5J is a plot of the uptake levels of microspheres in the maturated mBMDCs after a 15 min treatment of dark-red fluorescent microspheres; in which at least 200 cells were measured in each group per trial, and at least 1000 cells were measured in each group for five independent trials (n=5). The error bars represent the standard error of the mean. *p<0.05, **p<0.01, ***p<0.001, ****p<0.0001, one-way ANOVA with Tukey's HSD post-hoc test.

FIGS. 6A to 6I demonstrate that the NZ-maturated mBMDCs promote in vitro activation of antigen-specific CTLs into the PD-1^{low}CD44^{high} memory phenotypes. FIG. 6A is a schematic of the experimental timeline to analyze the properties of antigen-specific CTLs primed by the mBMDCs maturated with the silica NZs(-P245-N3) or LPS; in which the CTLs primed by the TCPS-attached mBMDCs served as the negative control group. FIG. 6B shows representative histograms of IFNγ in the antigen-specific H-2k^{b}-OVA₂₅₇₋₂₆₄-tetramer⁺ CD8⁺ CTL subsets after 4 days of DC-CTL co-culture and the treatment with GolgiPlug on the day of harvest (n=4). FIG. 6C is a plot of the geometric MFI of IFNγ in the antigen-specific H-2k^{b}-OVA₂₅₇₋₂₆₄-tetramer⁺ CD8⁺ CTL subsets after 4 days of DC-CTL co-culture and the treatment with GolgiPlug on the day of harvest (n=4). FIG. 6D shows representative histograms of CFSE^{low} divided cells in the antigen-specific H-2k^{b}-OVA₂₅₇₋₂₆₄-tetramer⁺ CD8⁺ CTL subsets after 4 days of DC-CTL co-culture; the lines on histograms indicate the gating for CFSE^{low} cells. FIG. 6E is a plot of the normalized percentage of CFSE^{low} divided cells in the antigen-specific H-2k^{b}-OVA₂₅₇₋₂₆₄-tetramer⁺ CD8⁺ CTL subsets after 4 days of DC-CTL co-culture; n=5; the percentage of CFSE^{low} CTLs is normalized by mean.

FIG. 6F shows representative histograms of CD44^{high} cells in the antigen-specific H-2k^{b}-OVA₂₅₇₋₂₆₄-tetramer⁺ CD8⁺ CTL subsets after 4 days of DC-CTL co-culture; the lines on histograms indicate the gating for CD44^{high} (F) cells. FIG. 6G is a plot of the normalized percentage of CD44^{high} cells in the antigen-specific H-2k^{b}-OVA₂₅₇₋₂₆₄-tetramer⁺ CD8⁺ CTL subsets after 4 days of DC-CTL co-culture; n=6. FIG. 6H shows representative histograms of PD-1 in the antigen-specific H-2k^{b}-OVA₂₅₇₋₂₆₄-tetramer⁺ CD8⁺ CTL subsets after 4 days of DC-CTL co-culture. FIG. 6I is a plot of the geometric MFI of PD-1 in the antigen-specific H-2k^{b}-OVA₂₅₇₋₂₆₄-tetramer⁺ CD8⁺ CTL subsets after 4 days of DC-CTL co-culture; n=6. The error bars represent the standard error of the mean. *p<0.05, **p<0.01, ***p<0.001, two-way ANOVA with Tukey's HSD post-hoc test.

FIGS. 7A to 7D illustrate the NZ-maturated mBMDCs suppress the growth rate of tumors in vivo. FIG. 7A is a schematic of the experimental timeline to examine the in vivo effect of the OVA₂₅₇₋₂₆₄-pulsed mBMDCs maturated with silica NZs(-P245-N3) or LPS on the mice inoculated with B16-OVA melanoma cells; in which the tumor-bearing mice injected with the vehicle (HBSS) without any mBMDCs served as the sham control group. FIG. 7B is a plot of the Kaplan-Meier survival curves of tumor-bearing mice after the injection of mBMDCs (log-rank test, p=0.0493, sham control versus NZ-maturated mBMDCs). FIG. 7C is a plot showing the growth rate of tumors in individual mice. FIG. 7D is plot of the summarized tumor volume of each experimental group (*sham control versus NZ-maturated mBMDCs, day 8: p=0.0507, day 10: p=0.0004, day 12: p<0.0001; #LPS-maturated mBMDCs versus NZ-maturated mBMDCs, day 10: p=0.0673, day 12: p=0.0123; † sham control versus LPS-maturated mBMDCs, day 8: p=0.0626, two-way ANOVA with Tukey's HSD post-hoc test). The error bars represent the standard error of the mean. n=9 for the sham control group, n=10 for the NZ-maturated mBMDCs and LPS-maturated mBMDCs, pooled from three independent trials.

FIGS. 8A to 8J demonstrate silica NZs promote the maturation of human monocyte-derived dendritic cells (hu-moDCs). FIG. 8A is a schematic of the experimental timeline to examine the properties of hu-moDCs maturated with silica NZs(-P245-N3); in which the hu-moDCs attached to the TCPS, without any maturation treatment, served as the negative control group; and the TCPS-attached hu-moDCs maturated with 100 ng m⁻¹ LPS for 20 h were used as the positive control for the validation of the assay method. FIG. 8B shows representative histograms of MHC-I, and MHC-II in the CD11c⁺ MHC-II⁺ hu-moDC subset after the 20 h maturation period; in which the lines on histograms mark the peaks in histograms of the unpulsed TCPS-attached hu-moDCs. FIG. 8C is a plot of the geometric MFI of MHC-I in the CD11c⁺ MHC-II⁺ hu-moDC subset after the 20 h maturation period (n=6). FIG. 8D is a plot of the geometric MFI of MHC-II in the CD11c⁺ MHC-II⁺ hu-moDC subset after the 20 h maturation period (n=8). FIG. 8E shows representative histograms of CD80, CD86, CCR7, XCR1 and DC-SIGN in the CD11c⁺ MHC-II⁺ hu-moDC subset after the 20 h maturation period; in which the lines on histograms mark the peaks in histograms of the unpulsed TCPS-attached hu-moDCs. FIG. 8F is a plot of the geometric MFI of CD80 in the CD11c⁺ MHC-II⁺ hu-moDC subset after the 20 h maturation period (n=6). FIG. 8G is a plot of the geometric MFI of CD86 in the CD11c⁺ MHC-II⁺ hu-moDC subset after the 20 h maturation period (n=5). FIG. 8H is a plot of the geometric MFI of CCR7 in the CD11c⁺ MHC-II⁺ hu-moDC subset after the 20 h maturation period (n=4). FIG. 8I is a plot of the geometric MFI of XCR1 in the CD11c⁺ MHC-II⁺ hu-moDC subset after the 20 h maturation period (n=4). FIG. 8J is a plot of the geometric MFI of DC-SIGN in the CD11c⁺ MHC-II⁺ hu-moDC subset after the 20 h maturation period (n=4). In FIGS. 8C, 8D, 8F to 8J, the error bars represent the standard error of the mean; *p<0.05, **p<0.01, ***p<0.001, ****p<0.0001, (TCPS-attached versus NZ-maturated hu-moDCs, with or without CEA pulsing) two-way ANOVA with Šídák's multiple comparisons test, (comparison among the hu-moDCs without CEA pulsing) Welch's ANOVA with Dunnett's T3 post-hoc test (C-D, F, H), one-way ANOVA with Tukey's HSD post-hoc test (G, I-J).

FIGS. 9A to 9J are SEM cross-sectional images of biocompatabile nanomatrices of various embodiments of the present invention, particularly they show silica nanomatrices composed of different nanostructures; scale bars: 250 nm. FIG. 9A is the SEM cross-section image of silica vertical nanopillars (vNPs) with a height (H) of 490 nm (i.e., vNPs-H490). FIG. 9B is the SEM cross-section image of silica tilted nanopillars (tNPs) with a H of 495 nm (i.e., tNPs-H495). FIG. 9C is the SEM cross-section image of left-handed silica nanohelices (NHs) with a pitch (P) of 245 nm and a number of pitch (N) of 2 (i.e., NHs-P245-N2-LH). FIG. 9D is the SEM cross-section image of right-handed silica NHs with a pitch (P) of 245 nm and a number of pitch (N) of 2 (i.e., NHs-P245-N2-RH). FIG. 9E is the SEM cross-section image of three-pitch silica nanozigzags (NZs) with a P of 165 nm (i.e., NZs-P165-N3). FIG. 9F is the SEM cross-section image of three-pitch silica NZs with a P of 245 nm (i.e., NZs-P245-N3).

FIG. 9G is the SEM cross-section image of three-pitch silica NZs with a P of 325 nm (i.e., NZs-P325-N3). FIG. 9H is the SEM cross-section image of silica NZs with a P of 245 nm and a N of 2, (i.e., NZs-P245-N2). FIG. 9I is the SEM cross-section image of silica NZs with a P of 245 nm and a N of 4 (i.e., NZs-P245-N4). FIG. 9J is the SEM cross-section image of silica NZs with a P of 245 nm and a N of 5 (i.e., NZs-P245-N5).

FIGS. 10A to 10D demonstrate the effects of silica nanomatrices with different shapes on the mBMDCs. FIG. 10A is a schematic illustration of silica nanomatrices composed of vertical nanopillars (vNPs-H490), nanohelices with different handedness (NHs-P245-N2-LH and NHs-P245-N2-RH), tilted nanopillars (tNPs-H495) and nanozigzags (NZs-P165-N3); in which the mBMDCs were applied to tissue culture polystyrene (TCPS) and silica nanomatrices composed of different shapes for 20 hours.

FIG. 10B is a plot of the percentage of CD86+ cells in the CD11c⁺ MHC-II⁺ mBMDC subset after the 20-hour maturation period (n=4). FIG. 10C is a plot of the percentage of DC-SIGN⁺ cells in the CD11c⁺ MHC-II⁺ mBMDC subset after the 20-hour maturation period (n=4). FIG. 10D is a plot of the percentage of XCR1+ cells in the CD11c⁺ MHC-II⁺ mBMDC subset after the 20-hour maturation period (n=4). In FIGS. 10B to 10D, the error bars represent standard error of mean. *p<0.05, **p<0.01, ****p<0.0001, one-way ANOVA with Tukey's HSD post-hoc test.

FIGS. 11A to 11D demonstrate the effects of pitch on the silica NZ-induced changes of protein expression in the mBMDCs. FIG. 11A is a schematic illustration of three-pitch silica NZs composed of controllable pitches (with a P of 245 and 325 nm, i.e., NZs-P245-N3 and NZs-P325-N3); the mBMDCs were applied to TCPS and silica NZs to maturate for 20 hours. FIG. 11B is a plot of geometric MFI of CD86 in the CD11c⁺ MHC-II⁺ mBMDCs after the 20-hour maturation period (n=4). FIG. 11C is a plot of geometric MFI of DC-SIGN in the CD11c⁺ MHC-II⁺ mBMDCs after the 20-hour maturation period (n=4). FIG. 11D is a plot of geometric MFI of XCR1 in the CD11c⁺ MHC-II⁺ mBMDCs after the 20-hour maturation period (n=4). In FIGS. 11B to 11D, the error bars represent standard error of mean. ***p<0.001, ****p<0.0001, one-way ANOVA with Tukey's HSD post-hoc test.

FIGS. 12A to 12F demonstrate the effects of silica NZs on the viability and expressions of co-stimulatory molecules and MHC molecules in mBMDCs; in which the mBMDCs were maturated with silica NZs(-P245-N3) or LPS for 20 hours; the mBMDCs attached to TCPS without any maturation treatment were served as the negative control group. FIG. 12A is a plot of the percentage of live cells, indicated by the absence of fixable viability dye, in the cell debris-excluded single-cell subsets (n=8).

FIG. 12B shows representative scatter plots of expressions of CD80 and CD86 in the CD11c⁺ MHC-II⁺ mBMDC subset after the 20-hour maturation period (n=6). FIG. 12C is a plot of the percentage of CD80+CD86+ cells in the CD11c⁺ MHC-II⁺ mBMDC subset after the 20-hour maturation period (n=6). FIG. 12D shows representative histograms of MHC-I and MHC-II in the CD11c⁺ MHC-II⁺ mBMDCs after the 20-hour maturation period. FIG. 12E is a plot of the geometric MFI of MHC-I in the CD11c⁺ MHC-II⁺ mBMDCs after the 20-hour maturation period; n=13. FIG. 12F is a plot of the geometric MFI of MHC-II in the CD11c⁺ MHC-II⁺ mBMDCs after the 20-hour maturation period; n=19. In FIGS. 12A, 12C, 12E and 12F, the error bars represent standard error of mean. *p<0.05, **p<0.01, ****p<0.0001, (A, C, F) one-way ANOVA with Tukey's HSD post-hoc test, (E) Welch's ANOVA with Dunnett's T3 post-hoc test.

FIGS. 13A to 13E demonstrate the effect of silica NZs on the production of pro-inflammatory cytokines in mBMDCs after the 20-hour maturation period. FIG. 13A is a schematic of experimental timeline to examine the intracellular level of cytokines in the maturated mBMDCs after the maturation period; in which the mBMDCs attached to the TCPS, without any maturation treatment, were served as the negative control group. FIG. 13B shows representative histograms of the intracellular level of pro-inflammatory cytokine TNFα, IFNγ and IL12-p40 in the CD11c⁺ MHC-II⁺ mBMDC subset after the 20-hour maturation period and the subsequent treatment with GolgiPlug. FIG. 13C is a plot of the geometric MFI of the intracellular level of pro-inflammatory cytokine TNFα in the CD11c⁺ MHC-II⁺ mBMDC subset after the 20-hour maturation period and the subsequent treatment with GolgiPlug (n=5). FIG. 13D is a plot of the geometric MFI of the intracellular level of pro-inflammatory cytokine IFNγ in the CD11c⁺ MHC-II⁺ mBMDC subset after the 20-hour maturation period and the subsequent treatment with GolgiPlug (n=4). FIG. 13E is a plot of the geometric MFI of the intracellular level of pro-inflammatory cytokine IL12-p40 in the CD11c⁺ MHC-II⁺ mBMDC subset after the 20-hour maturation period and the subsequent treatment with GolgiPlug (n=5). In FIGS. 13C to 13E, the error bars represent standard error of mean. ***p<0.001, ****p<0.0001, one-way ANOVA with Tukey's HSD post-hoc test.

FIG. 14 shows top-down SEM images of nanostructures of silica NZs, including NZs-P245-N3, NZs-P245-N4, NZs-P245-N5 at the left column (scale bars: 1 μm); and their sum-projected deconvoluted confocal images of the CellTracker Deep Red dye-stained NZs (scale bars: 5 μm).

FIG. 15 shows localization of vinculin and PIP2 in the NZ-maturated mBMDCs, characterized with confocal imaging. After a 20-hour maturation with the silica NZs-P245-N3, the mBMDCs were fixed and immuno-stained for confocal imaging: magenta for vinculin and green for PIP2.

FIG. 16 is a diagram showing properties of cell adhesions in the mBMDCs attached to silica NZs(-P245-N3) for 2 hours, 4 hours and 20 hours. The representative maximum intensity projections of confocal images show that the mBMDCs attached to silica NZs for 20 hours have more abundant vinculin⁺ cell adhesions, compared with the mBMDCs attached to silica NZs for 2 or 4 hours. The yellow outlines of cell are defined by the staining of F-actin (scale bars: 5 μm). The orthogonal views in the YZ and XZplanes labelled with the dotted lines show that the depth of cell adhesions (indicated by the white brackets) increased with the time of cell attachment (scale bars: 2 μm). These results illustrate that the mBMDCs have a more extensive formation of cell adhesions after 20 hours of cell attachment.

FIGS. 17A to 17D demonstrate the effect of FAK inhibitor PF-573228 on the expression of co-stimulatory molecules and CD206 in the silica NZs(-P245-N3)-maturated mBMDCs, in which the experimental timeline of NZ-mediated maturation and treatment with PF-573228 (PF) is indicated in FIG. 5A. FIG. 17A shows the representative scatter plots of CD80⁺ CD86⁺ cells in the CD11c⁺ MHC-II⁺ mBMDC subset after the 20-hour maturation period. FIG. 17B is a plot showing the percentage of CD80⁺ CD86⁺ cells in the CD11c⁺ MHC-II⁺ mBMDC subset after the 20-hour maturation period (n=4). FIG. 17C is a histogram of CD206 cells in the CD11c⁺ MHC-II⁺ mBMDC subset after the 20-hour maturation period. FIG. 17D is a plot of CD206⁺ cells in the CD11c⁺ MHC-II⁺ mBMDC subset after the 20-hour maturation period (n=3). In FIGS. 17B and 17D, the error bars represent standard error of mean. ns (non-significant), **p<0.01, ***p<0.001, one-way ANOVA with Tukey's HSD post-hoc test.

FIGS. 18A to 18E demonstrate the effect of FAK inhibitor PF-573228 on the production of pro-inflammatory cytokines in the silica NZs(-P245-N3)-maturated mBMDCs. FIG. 18A is a schematic of experimental timeline to examine the effect of PF-573228 on the NZ-enhanced intracellular level of cytokines in mBMDCs after the maturation period; in which the mBMDCs attached to TCPS, with the treatment of 0.02% DMSO, were served as the negative control group. FIG. 18B shows representative histograms of the intracellular level of TNFα, IFNγ and IL12-p40 in the CD11c⁺ MHC-II⁺ mBMDC subset after the 20-hour maturation period and the subsequent treatment with GolgiPlug. FIG. 18C is a plot of the geometric MFI of the intracellular level of TNFα in the CD11c⁺ MHC-II⁺ mBMDC subset after the 20-hour maturation period and the subsequent treatment with GolgiPlug (n=5). FIG. 18D is a plot of the geometric MFI of the intracellular level of IFNγ in the CD11c⁺ MHC-II⁺ mBMDC subset after the 20-hour maturation period and the subsequent treatment with GolgiPlug (n=4). FIG. 18E is a plot of the geometric MFI of the intracellular level of IL12-p40 in the CD11c⁺ MHC-II⁺ mBMDC subset after the 20-hour maturation period and the subsequent treatment with GolgiPlug (n=5). In FIGS. 18C to 18E, the error bars represent standard error of mean. *p<0.05, **p<0.01, ***p<0.001, ****p<0.0001, one-way ANOVA with Tukey's HSD post-hoc test.

FIGS. 19A to 19C demonstrate abundance of cell adhesions with active FAK in the mBMDCs maturated with silica NZs composed of different P and N. FIG. 19A shows representative confocal images which show the immunostaining results of vinculin and p-FAK (Y397) at the basal plasma membranes of mBMDCs attached to TCPS and silica NZs for 20 hours. FIG. 19B is a plot of the percentage of cell area covered with vinculin⁺ cell adhesions in the mBMDCs attached to TCPS (n=140 cells), NZs-P245-N2 (n=140 cells), NZs-P165-N3 (n=142 cells) and NZs-P245-N3 (n=156 cells), pooled from two independent trials. FIG. 19C is a plot of the fluorescence intensities of p-FAK in the vinculin⁺ cell adhesions in the mBMDCs attached to TCPS (n=140 cells), NZs-P245-N2 (n=140 cells), NZs-P165-N3 (n=142 cells) and NZs-P245-N3 (n=156 cells), pooled from two independent trials. In FIGS. 19B and 19C, the error bars represent standard error of mean. **p<0.01, ****p<0.0001, Welch's ANOVA with Games-Howell's post-hoc test.

FIGS. 20A to 20D demonstrate further analysis of CFSE-based CTL proliferation results in the antigen-specific CTLs primed by the mBMDCs for 4 days; these results are supplementary to the results of CFSE proliferation assay presented in FIGS. 6D and 6E. FIG. 20A shows representative histograms and gating strategies for analyzing the CFSE-indicated cell divisions in the H-2k^{b}-OVA₂₅₇₋₂₆₄-tetramer⁺ antigen-specific CTLs; in which the phytohemagglutinin-L (PHA-L)-treated CTLs were used as the positive control for defining the gating for each cell division. FIG. 20B is a plot of the percentage of H-2k^{b}-OVA₂₅₇₋₂₆₄-tetramer⁺ antigen-specific CTLs with more than one cell division (n=5). FIG. 20C is a plot of the percentage of H-2k^{b}-OVA₂₅₇₋₂₆₄-tetramer⁺ antigen-specific CTLs with more than two cell divisions (n=5). FIG. 20D is a plot of the percentage of H-2k^{b}-OVA₂₅₇₋₂₆₄-tetramer⁺ antigen-specific CTLs with more than three cell divisions (n=5). The results in FIGS. 20B to 20D are normalized by mean and presented as the fold change relative to the mean of CTLs primed by the unpulsed TCPS-attached mBMDCs. The error bars represent standard error of mean. *p<0.05, **p<0.01, ****p<0.0001, two-way ANOVA with Tukey's HSD post-hoc test.

FIGS. 21A to 21H demonstrate the expressions of surface proteins and cytokines in the mBMDCs pulsed with OVA₂₅₇₋₂₆₄ peptide. FIG. 21A is a schematic of experimental timeline to characterize the properties of NZs(-P245-N3)-maturated mBMDCs and LPS-maturated mBMDCs pulsed with OVA₂₅₇₋₂₆₄ peptide; in which the TCPS-attached mBMDCs without any maturation treatment were served as the negative control group. FIG. 21B is a representative histogram of OVA₂₅₇₋₂₆₄-bound MHC-I (H-2k^{b}-OVA₂₅₇₋₂₆₄) in the CD11c⁺ MHC-II⁺ mBMDCs pulsed with OVA₂₅₇₋₂₆₄ peptide for 2 hours; wherein the light-grey histogram represents the unstained control. FIG. 21C is a plot of the geometric MFI of OVA₂₅₇₋₂₆₄-bound MHC-I (H-2k^{b}-OVA₂₅₇₋₂₆₄) in the CD11c⁺ MHC-II⁺ mBMDCs pulsed with OVA₂₅₇₋₂₆₄ peptide for 2 hours. FIG. 21D is a representative histogram of CD86 in the H-2k^{b}-OVA₂₅₇₋₂₆₄-expressing mBMDCs pulsed with OVA₂₅₇₋₂₆₄ peptide for 2 hours.

FIG. 21E is a plot of the geometric MFI of CD86 in the H-2k^{b}-OVA₂₅₇₋₂₆₄-expressing mBMDCs pulsed with OVA₂₅₇₋₂₆₄ peptide for 2 hours. FIG. 21F is a plot of the geometric MFI of intracellular level of TNFα in the CD11c⁺ MHC-II⁺ mBMDCs pulsed with OVA₂₅₇₋₂₆₄ peptide for 2 hours and subsequently treated with GolgiPlug. FIG. 21G is a plot of the geometric MFI of intracellular level of IFNγ in the CD11c⁺ MHC-II⁺ mBMDCs pulsed with OVA₂₅₇₋₂₆₄ peptide for 2 hours and subsequently treated with GolgiPlug. FIG. 21H is a plot of the geometric MFI of intracellular level of IL12-p40 in the CD11c⁺ MHC-II⁺ mBMDCs pulsed with OVA₂₅₇₋₂₆₄ peptide for 2 hours and subsequently treated with GolgiPlug. These results together show that the pulsing of OVA₂₅₇₋₂₆₄ peptide did not further enhance the expressions of these cytokines in the NZ-maturated mBMDCs and LPS-maturated mBMDCs. The error bars represent standard error of mean. a.u. (arbitrary unit), n=4, *p<0.05, **p<0.01, ***p<0.001, ****p<0.0001, (C, E) one-way ANOVA with Tukey's HSD post-hoc test, (F-H) two-way ANOVA with Tukey's HSD post-hoc test.

FIGS. 22At to 22F demonstrate expressions of T-bet, CD44 and PD-1 in the antigen-specific CTLs primed by the mBMDCs for 4 days; in which the experimental timeline to prime the antigen-specific CTLs with the mBMDCs maturated with silica NZs(-P245-N3) or LPS is indicated in FIG. 6A, and the CTLs primed by the TCPS-attached mBMDCs were serve as the negative control group. FIG. 22A shows representative histograms and gating of H-2k^{b}-OVA₂₅₇₋₂₆₄-tetramer⁺ antigen-specific CTLs with high expression level of T-bet (T-bet^{high}). FIG. 22B is a plot of the percentage of H-2k^{b}-OVA₂₅₇₋₂₆₄-tetramer⁺ antigen-specific CTLs with high expression level of T-bet (T-bet^{high}). FIG. 22C shows representative histograms of CD44 in the T-bet^{high} H-2k^{b}-OVA₂₅₇₋₂₆₄-tetramer⁺ antigen-specific CTLs; the line on histograms marks the peak in histogram of CTLs primed by the unpulsed TCPS-attached mBMDCs.

FIG. 22D is a plot of the geometric MFI of CD44 in the T-bet^{high} H-2k^{b}-OVA₂₅₇₋₂₆₄-tetramer⁺ antigen-specific CTLs. FIG. 22E shows representative histograms of PD-1 in the H-2k^{b}-OVA₂₅₇₋₂₆₄-tetramer⁺ antigen-specific CTLs with intermediate expression level of T-bet (T-bet^{int}). FIG. 22F is a plot showing the geometric MFI of PD-1 in the H-2k^{b}-OVA₂₅₇₋₂₆₄-tetramer⁺ antigen-specific CTLs with intermediate expression level of T-bet (T-bet^{int}). In FIGS. 22B, 22D, 22F, the error bars represent standard error of mean. n=4, *p<0.05, **p<0.01, ***p<0.001, ****p<0.0001, two-way ANOVA with Tukey's HSD post-hoc test.

FIGS. 23A to 23C demonstrate the effect of silica NZs(-P245-N3)-maturated hu-moDCs on activating the human CTLs. FIG. 23A is a schematic of experimental timeline to examine the degranulation marker (CD107a) and production of effector protein IFNγ in the CTLs co-cultured with the maturated hu-moDCs for 9 days. FIG. 23B shows representative scatter plots of CD107a⁺IFNγ⁺ CTLs in the CD8⁺ CTL subset (n=6) demonstrate the capacity of NZ-maturated hu-moDCs to promote the non-antigen-specific activation of CTLs. FIG. 23C is a plot of CD107a⁺IFNγ⁺ CTLs in the CD8⁺ CTL subset (n=6) demonstrate the capacity of NZ-maturated hu-moDCs to promote the non-antigen-specific activation of CTLs. The error bars represent standard error of mean. **p<0.01, ***p<0.001, two-way ANOVA with Šídák's multiple comparisons test.

FIG. 24A is a plot showing the uptake of dextran in the TCPS-attached mBMDCs and NZ(-P245-N3)-maturated mBMDCs at 37° C. and 4° C.

FIG. 24B is a plot showing the uptake of microspheres in the TCPS-attached mBMDCs and NZ(-P245-N3)-maturated mBMDCs at 37° C. and 4° C. In these uptake analysis, the mBMDCs were treated with AF647-conjugated dextran or dark-red fluorescent microspheres at 37° C. and 4° C. for 15 minutes. Afterward, the cells were fixed, counterstained with phalloidin and quantified using the confocal microscopy. At least 30 observation fields were measured for each group per trial. The results confirmed that the intracellular dextran in NZ-maturated mBMDCs and the intracellular microspheres in the TCPS-attached mBMDCs and NZ-maturated mBMDCs were internalized via endocytosis. The error bars represent standard error of mean. n=3, **p<0.01, ****p<0.0001, two-tailed Student's t-test.

### DETAILED DESCRIPTION OF THE PREFERRED EMBODIMENTS

Unless otherwise defined, all technical terms used herein have the same meaning as commonly understood by one skilled in the art to which the invention belongs.

As used herein, “comprising” means including the following elements but not excluding others. “Essentially consisting of” means that the material consists of the respective element along with usual and unavoidable impurities such as side products and components usually resulting from the respective preparation or method for obtaining the material such as traces of further components or solvents. “Consisting of” means that the material solely consists of, i.e. is formed by the respective element. As used herein, the forms “a”, “an”, and “the”, are intended to include the singular and plural forms unless the context clearly indicates otherwise.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

As used herein, the term “effective amount” refers to the amount of the composition sufficient to effect beneficial or desired results. An effective amount can be administered in one or more administrations, applications or dosages and is not intended to be limited to a particular formulation or administration route.

As used herein, and unless otherwise indicated, the terms “treating”, “treatment” and the like refer to alleviating or reducing the severity of a disease or a symptom associated with the disease or condition being treated. The terms “prevent”, “prevention” and other forms of the word include the inhibition of onset or progression of a disease or disorder or a symptom of the particular disease or disorder.

### I. Methods of Producing Matured Dendritic Cells

In one aspect, the invention provides a method for producing matured dendritic cells (DCs) for immunotherapy. Dendritic cells (DCs), as used herein, refers to antigen-presenting cells of the immune system that are characterized by their ability to process and present antigens to T lymphocytes. DCs are distinguished by their morphology, including branched cytoplasmic projections, and by their functional role in initiating and regulating adaptive immune responses. Immature DCs are capable of antigen uptake and processing, whereas matured DCs express co-stimulatory molecules and major histocompatibility complex (MHC) class I and class II molecules, thereby enabling activation of cytotoxic and helper T cells. The immature DCs are selected from bone marrow-derived DCs, monocyte-derived DCs, or CD34⁺ hematopoietic stem cells. In some instances, the immature DCs are monocyte-derived DCs, or bone marrow-derived DCs.

In particular, the methods of the present invention comprise the following steps:
a) providing a biocompatible nanomatrix;
b) seeding immature DCs onto the biocompatible nanomatrix; and
c) culturing the DCs for a period sufficient to induce maturation via mechanical stimuli provided by the biocompatible nanomatrix.

The “biocompatible nanomatrix” refers to a nanoscale structured material or scaffold that is capable of interacting with biological systems without eliciting significant adverse immune or toxic responses. A biocompatible nanomatrix may comprise polymers, lipids, proteins, inorganic materials, or combinations thereof, arranged in a nanoscale architecture that supports cell adhesion, growth, differentiation, or functional activity. In embodiments, the nanomatrix is designed to facilitate the maturation of dendritic cells, the presentation of antigens, or the delivery of therapeutic agents, while maintaining compatibility with physiological environments. In particular, the nanomatrix comprises a biocompatible material including, but not limited to, silica, polyethylene (PE), polytetraflurorethylene (PTFE), poly(lactic acid) (PLA), poly(glycolic acid) (PGA), polycaprolactone (PCL), hydrogels, collagen, chitosan, alginate, titanium, titanium alloy, stainless steel, hydroxyapatite, or the like.

In embodiments, the nanomatrix comprises a substrate, and a plurality of nanostructures protruding from the substrate. The substrate may be selected from biocompatible or functional materials that provide mechanical stability and electrical conductivity, including but not limited to indium tin oxide (ITO)-coated glass slides, silicon wafers, or other conductive or semiconductive surfaces.

The nanostructures protruding from the substrate may be composed of silica, including amorphous or mesoporous silica, fabricated to provide controlled dimensions such as diameters ranging from about 10 nm to about 1000 nm and lengths ranging from about 10 nm to 1000 nm. The nanostructures may be arranged in ordered or disordered arrays, and may take the form of nanofibers, nanorods, nanotubes, nanopillars or nanozigzags extending from the substrate.

In some instances, the nanostructures are composed of silica. In certain embodiments, the nanostructures are configured in a zigzag geometry, such that they form nanozigzags extending from the substrate. This configuration provides a unique nanoscale topography that increases surface area and mechanical interaction with cells, thereby promoting adhesion and facilitating the maturation of dendritic cells. The nanozigzag arrangement may further enhance antigen presentation efficiency and improve the immunostimulatory capacity of the matured DCs. In particular, the zigzag geometry is configured to induce mechanical maturation of dendritic cells via activation of focal adhesion kinase.

In embodiments, each of the nanostructures comprises between two and five pitches. It has been observed that nanostructures configured with three to five pitches induce a notable enhancement in the expression levels of DC maturation markers, including CD86 and DC-SIGN. Without being bound by theory, it is believed that the repeating zigzag geometry of the pitches provides increased nanoscale surface area and mechanical cues that promote DC adhesion and activation, thereby facilitating functional maturation. In certain embodiments, each of the nanostructures comprises three pitches, or at least three pitches. The nanostructures with three pitches have demonstrated reproducible enhancement in dendritic cell maturation, providing a balance between structural stability and biological activity.

In embodiments, each of the pitches has a height of approximately 100 nm to 500 nm, measured as the vertical distance between successive vertices of identical orientation within the repeating zigzag arrangement. Preferably, the pitch height is within the range of 100 nm to 200 nm or within the range of 200 nm to 400 nm, and more specifically may be about 165 nm, about 245 nm, or about 325 nm. In certain preferred embodiments, the pitch height is about 245 nm, with a tolerance of ±10 nm or ±5 nm. Such dimensional control provides optimized nanoscale cues that enhance DC maturation and antigen presentation efficiency.

In certain embodiments, the nanostructures are fabricated using glancing angle deposition (GLAD). GLAD is a physical vapor deposition technique in which a vapor flux of material, such as silica, is directed toward a substrate at an oblique angle relative to the substrate surface. By controlling the deposition angle, substrate rotation, and deposition rate, nanostructures with tailored geometries—including zigzag, helical, or columnar forms—can be reproducibly generated. In some instances, the GLAD process is employed to fabricate nanozigzags comprising multiple pitches, wherein the repeating angular deposition pattern produces the desired zigzag architecture. This method allows precise control over nanostructure dimensions, orientation, and density, thereby enabling the creation of nanomatrices optimized for DC adhesion, maturation, and antigen presentation. In some instances, the fabrication may follow the protocols described in J. Deng, Z. Huang, RSC Adv. 2016, 6, 84348, and Z. Huang, et al., Nanotechnology 2012, 23, 275703.

In embodiments, the biocompatible nanomatrix is configured to upregulate the expression of key DC maturation markers, including CD86, DC-SIGN, XCL1, CCR7, IL-12, and/or TNFα. The nanomatrix may also enhance the endocytic capacity of the dendritic cells, thereby improving their ability to internalize and process antigens. In some instances, the matured DCs produced according to the methods disclosed herein retain elevated levels of C-type lectin receptors (CLRs) and maintain high endocytic capacity even after the maturation period, for example, after approximately 10 hours, 15 hours, or 20 hours. This sustained endocytic activity enables more efficient antigen loading and presentation. As a result, CTLs are activated more effectively, with prolonged T-cell receptor engagement and co-stimulatory signaling, thereby supporting their effector function, and differentiation into long-lived memory T cells.

In embodiments, the period sufficient to induce maturation of the DCs ranges from approximately 30 minutes to 48 hours, such as from about 1 hour to 36 hours, or from about 12 hours to 24 hours. In some instances, the maturation period may be approximately 12, 13, 14, 15, 16, 17, 18, 19, 20, 21, 22, 23, or 24 hours. In particular embodiments, the period sufficient to induce maturation of the DCs is approximately 20 hours. This time frame has been observed to allow the DCs to undergo phenotypic and functional changes characteristic of maturation, including upregulation of surface markers and enhanced antigen-presenting capacity. The defined period provides reproducibility and consistency in generating matured DCs suitable for therapeutic use.

In preferred embodiments, the maturation step is performed in the absence of exogenous chemical maturation agents or cytokines. This approach avoids the use of external stimulants such as TNF-α, IL-1β, or lipopolysaccharide, thereby reducing variability and potential adverse effects associated with chemical induction. The physical and structural cues provided by the nanomatrix are sufficient to promote DCs maturation under these conditions.

In embodiments, the matured DCs are configured to activate antigen-specific cytotoxic T-lymphocytes (CTLs). The matured dendritic cells present tumor-associated or pathogen-derived antigens in the context of MHC molecules, thereby priming CTLs to recognize and eliminate target cells. This activation is a critical step in eliciting a robust adaptive immune response.

In specific embodiments, the activated antigen-specific CTLs exhibit a PD-1^{low}CD44^{high} memory phenotype. Such a phenotype is indicative of a relatively long-lived memory population with reduced susceptibility to exhaustion. In some instances, the PD-1^{low}CD44^{high} CTLs can be rescued from apoptosis and may restore their proliferative capacity and effector functions through the application of immune checkpoint inhibitors. Accordingly, the presence of this phenotype may be suitable for enhancing therapeutic outcomes in combinational treatment strategies, for example, in conjunction with immune checkpoint blockade therapies.

In embodiments, the method further comprises pulsing the DCs with at least one tumor-associated antigen prior to administration. The antigen may be selected from peptides, proteins, nucleic acids, or tumor lysates, and may be derived from known tumor-associated antigens such as carcinoembryonic antigen (CEA), Mucin 1 (MUC1), human epidermal growth factor receptor 2 (HER2/neu), or glycoprotein 100 (gp100). Pulsing the dendritic cells with tumor-associated antigens enhances their ability to prime antigen-specific cytotoxic T-lymphocytes and to elicit targeted anti-tumor immune responses.

It will be appreciated that the methods disclosed herein are suitable for the preparation of DC-based cancer vaccines. Accordingly, the invention also provides methods for preparing DC-based cancer vaccines, including the methods described above, and preferably further comprising one or more steps directed to activating the matured DCs. In some instances, the matured DCs prepared according to the methods disclosed above are co-cultured with antigen-specific cytotoxic T-lymphocytes (CTLs). During co-culture, the matured DCs present processed tumor-associated antigens in the context of MHC molecules and provide co-stimulatory signals through upregulated molecules such as CD86, DC-SIGN, and CCR7. This interaction promotes efficient activation of CTLs, resulting in clonal expansion, enhanced effector function, and differentiation into long-lived memory T cells. In some instances, the co-culture conditions may be optimized to sustain prolonged T-cell receptor engagement and cytokine signaling, thereby improving the immunological potency of the CTLs. The co-cultured CTLs may subsequently be harvested and administered to a subject as part of an adoptive cell transfer therapy, or used in combination with dendritic cell vaccines to augment anti-tumor immune responses.

### II. Matured DCs

The invention also provides the matured DCs produced according to the disclosed methods. The matured DCs produced according to the disclosed methods would be suitable for immunotherapy, particularly to modulate, enhance, or harness the immune system of a subject in need thereof to prevent, treat, or control a disease. In particular, the matured DCs could be used to formulate a DC-based cancer vaccine for activating cytotoxic or helper T lymphocytes, thereby eliciting an anti-tumor immune response.

In some instances, the matured DCs are administered to the same subject from whom the immature DCs were originally obtained. In other instances, the composition may be formulated for allogeneic use, wherein the matured DCs are derived from a donor and administered to a different recipient.

In embodiments, the matured DCs are configured to activate antigen-specific CTLs. The matured DCs present tumor-associated or pathogen-derived antigens in the context of MHC molecules, thereby priming CTLs to recognize and eliminate target cells. This activation is a critical step in eliciting a robust adaptive immune response.

In certain embodiments, the matured DCs exhibit upregulated expression of co-stimulatory and immunoregulatory molecules. Such molecules may include CD86, dendritic cell-specific ICAM-3 grabbing nonintegrin (DC-SIGN), X-C motif chemokine receptor 1 (XCR1), C-C chemokine receptor type 7 (CCR7), interleukin-12 (IL-12), and tumor necrosis factor-alpha (TNFα). The enhanced expression of these markers reflects the functional maturation of the DCs and contributes to their capacity to activate antigen-specific cytotoxic T-lymphocytes and to promote robust immune responses.

In specific embodiments, the activated antigen-specific CTLs exhibit a PD-1^{low}CD44^{high} memory phenotype. Such a phenotype is indicative of a relatively long-lived memory population with reduced susceptibility to exhaustion.

### III. Pharmaceutical Compositions

The present invention further provides pharmaceutical compositions comprising the matured DCs suitable for administration to a subject in need of immunotherapy. Preferably, the compositions comprise an effective amount of the matured DCs. Such compositions may include, in addition to the matured DCs, one or more pharmaceutically acceptable carriers, excipients, stabilizers, or adjuvants that maintain cell viability and functionality. In some embodiments, the pharmaceutical composition is formulated for autologous administration, wherein the matured DCs are derived from immature DCs isolated from the same subject. In other embodiments, the composition may be formulated for allogeneic use. The pharmaceutical compositions may be provided in liquid suspension, cryopreserved form, or encapsulated within a biocompatible matrix, and are intended to elicit or enhance an immune response, including but not limited to an anti-tumor response. The compositions may be formulated based on the intended route of administration.

The pharmaceutical compositions can contain one or more pharmaceutically acceptable carriers and/or one or more pharmaceutically acceptable excipients. Suitable pharmaceutically acceptable carriers and excipients are generally recognized as safe (GRAS), and may be administered to an individual without causing undesirable biological side effects or unwanted interactions. Suitable carriers include aqueous solutions such as physiological saline, phosphate-buffered saline (PBS), Ringer's solution, or culture media supplemented with serum proteins. In certain embodiments, the carrier may comprise cryopreservation media containing dimethyl sulfoxide (DMSO), glycerol, or other stabilizing agents to preserve dendritic cell functionality during freezing and thawing.

Suitable excipients include proteins such as human serum albumin, sugars such as trehalose or sucrose, and polymers such as polyethylene glycol (PEG) or polyvinylpyrrolidone (PVP). These excipients may act as osmoprotectants, cryoprotectants, or stabilizers to reduce cellular stress during formulation and administration.

In some embodiments, the pharmaceutical compositions may include immunological adjuvants or supportive agents that enhance the immune response elicited by the matured DCs. Examples include cytokines such as interleukin-2 (IL-2), granulocyte-macrophage colony-stimulating factor (GM-CSF), or toll-like receptor (TLR) agonists. These agents may be co-formulated or co-administered to potentiate antigen presentation and T cell activation.

It would be appreciated that the carriers, excipients, and/or adjuvants are selected to be compatible with human administration, non-toxic, and non-immunogenic, while preserving the biological activity of the DCs. The compositions may be formulated as injectable suspensions, cryopreserved vials, or encapsulated within biocompatible matrices such as hydrogels or liposomes, depending on the intended route of administration.

In embodiments where the pharmaceutical compositions are formulated as DC-based cancer vaccines, such compositions can be formulated for intradermal administration, subcutaneous administration, intravenous administration, intranodal administration, or intratumoral administration. In some instances, the compositions are formulated for intradermal or intranodal administration to elicit stronger T-cell responses.

The “effective amount of the matured DCs” refers to a quantity sufficient to elicit a measurable immunological response in a subject, such as activation of cytotoxic T lymphocytes, helper T cells, or induction of an anti-tumor immune response. The effective amount may vary depending on factors including the source of the DCs, the method of maturation, the route of administration, and the condition being treated. In particular, the effective amount comprises a population of matured DCs administered in a pharmaceutically acceptable carrier, wherein the number of cells is sufficient to achieve the intended therapeutic outcome without causing undue adverse effects.

In some instances, the matured DCs present at least one tumor-associated antigen on the MHC-I complexes, enabling recognition and activation of cytotoxic T lymphocytes.

In certain embodiments, the matured DCs exhibit upregulated expression of a co-stimulatory and immunoregulatory molecule selected from the group consisting of CD86, DC-SIGN, XCR1, CCR7, IL-12, TNFα, and a combination thereof.

### III. Treatment of Cancer

Methods of using the matured DCs or the pharmaceutical compositions for treating a cancer in a subject in need thereof are disclosed. Generally, the method includes (i) administering to the subject the pharmaceutical composition described above. The administration step can occur one or more times.

The subject can be a mammal, such as a human, a dog, a cat, a rat, a monkey, rabbits, guinea pigs, etc., that is in need of cancer treatment. In some instances, the subject can be exhibiting symptoms of or diagnosed with cancer.

The pharmaceutical composition can be administered by oral administration, parenteral administration, mucosal administration, or topical administration, or a combination thereof. In the preferred embodiments, the pharmaceutical compositions are formulated as DC-based cancer vaccines, such compositions can be formulated for intradermal administration, subcutaneous administration, intravenous administration, intranodal administration, or intratumoral administration.

The cancer being treated using the disclosed methods can be tumors, such as tumors of the hematopoietic and lymphoid tissues or hematopoietic and lymphoid malignancies, tumors that affect the blood, bone marrow, lymph, and lymphatic system, and tumors located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax, and genito-urinary apparatus. In some instances, the cancer can be colon cancer, pancreatic cancer, prostate cancer, breast cancer, ovarian cancer, lung cancer (e.g. non-small cell lung cancer), skin cancer (melanoma), brain cancer, leukemia (e.g. acute myeloid leukemia (AML) and chronic myeloid leukemia (CML)), kidney cancer, cervical cancer, liver cancer, or leukemia, or a combination thereof.

In some instances, the cancer can be melanoma, prostate cancer, glioblastoma, renal cell carcinoma, breast cancer, ovarian cancer, non-small cell lung cancer, colorectal cancer, pancreatic cancer, or leukemia. These cancers are characterized by the presence of tumor-associated antigens that can be effectively processed and presented by DCs to elicit antigen-specific CTL responses. In some instances, the matured DCs or the pharmaceutical compositions disclosed herein may be administered alone or in combination with other immunotherapeutic agents, such as immune checkpoint inhibitors, to enhance anti-tumor efficacy and promote durable immunological memory.

Accordingly, it would be appreciated that the invention also provides methods of treating a cancer in a subject in need thereof, by administering the matured DCs or the pharmaceutical compositions disclosed herein to the subject.

### EXAMPLES

### Example 1

### Biocompatible Nanomatrix for Producing Matured DCs

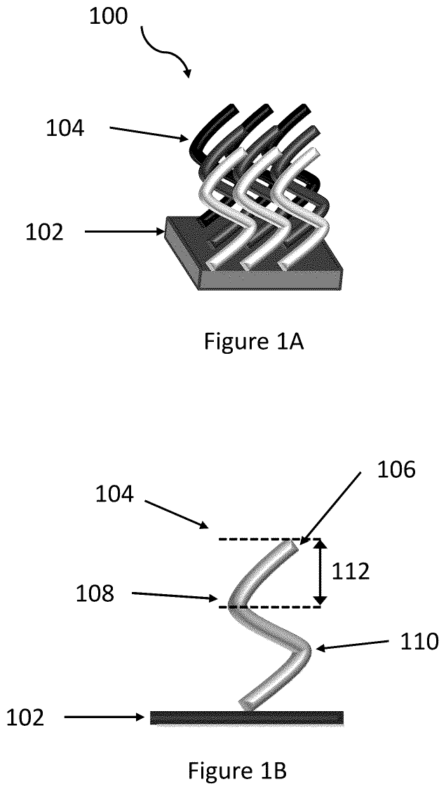

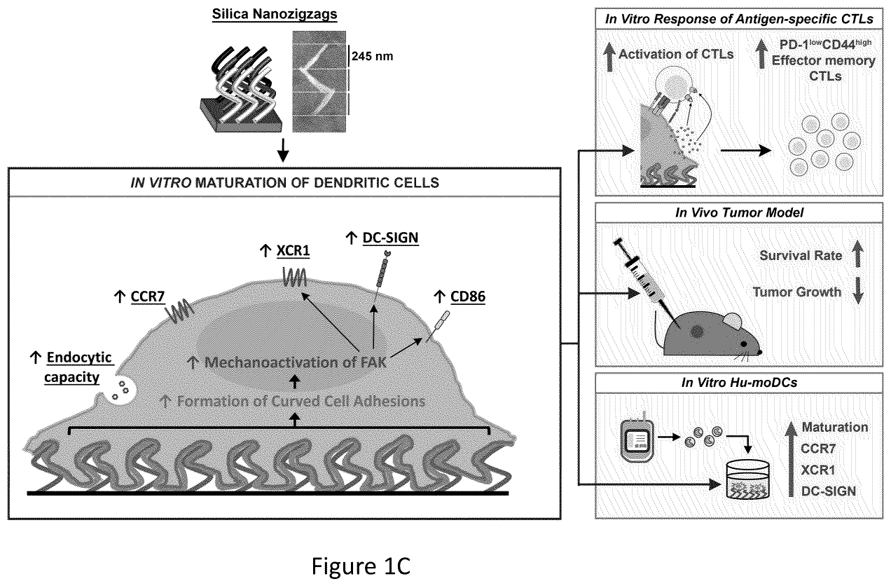

FIG. 1A illustrates an embodiment of a biocompatible nanomatrix 100 fabricated for use in the present invention. As shown in FIG. 1A, the biocompatible nanomatrix 100 includes a substrate 102, and a plurality of nanostructures 104 protruding from the substrate. With reference to FIG. 1B, the nanostructure 104 is preferably made of silica and is configured in zigzag geometry. The nanostructure 104 is therefore a nanozigzag (NZ) having at least one vertex and at least two pitches (P). In this particular embodiment, the NZ 104 has three vertices 106, 108 and 110, as well as three pitches (P). The height of P 112 can be between 100 nm to 500 nm. For example, the height of P can be 165 nm, or 245 nm.

As shown in FIG. 1C, there is illustrated a biocompatible nanomatrix comprising silica NZs (with a pitch (P) of 245 nm and the number pitch (N) of 3, i.e., P245-N3). The nanomatrix promotes the maturation of DCs into a phenotype that can potentially enhance their uptake of antigens, activation of cytotoxic T-lymphocytes (CTLs) and homing to lymph nodes, via the mechanoactivation of FAK initiated by the formation of curved cell adhesions at the DC-NZ interfaces. The NZ-maturated murine bone mBMDCs can activate the antigen-specific CTLs and program them into the PD-1^{low}CD44^{high} effector memory CTLs in vitro. Injection of the NZ-maturated mBMDCs suppresses the growth of tumors in vivo. The silica NZs are also applicable to the in vitro maturation of human monocyte-derived DCs (Hu-moDCs).

### Methods and Materials

### Fabrication and Characterization of Extracellular Silica Nanomatrices:

Glancing angle deposition (GLAD) was performed to produce silica nanomatrices vertically protruding on a supporting substrate. The detailed protocol of GLAD is as described in J. Deng, Z. Huang, RSC Adv. 2016, 6, 84348; and Z. Huang, et al., Nanotechnology 2012, 23, 275703. Specifically, using a custom-built physical vapor deposition system equipped with the high vacuum chamber (10⁻⁷-10⁻⁶ Torr, JunSun Tech Co. Ltd., Taiwan), silica (SiO₂) was evaporated at a rate of ≈0.4 nm s⁻¹ with electron-beam accelerating voltage of 8.0 kV and emission current of 83-87 mA. Silica was deposited on indium tin oxide (ITO)-coated glasses or silicon wafers, at a deposition angle (α) of 87° with respect to the substrate's normal direction. The deposition process was monitored using a quartz crystal microbalance located in the vicinity of the sample. An ethanol-water cooling system was used to maintain the substrate temperature at room temperature during GLAD. To sculpt silica in the shape of NZs, the substrate was rotated back and forth by 180° at a given time interval, with which the P of silica NZs was controlled. To produce the tilted nanorods, silica was deposited at α of 87° without rotating the substrate, see W. F. Lau, et al., Nanotechnology 2013, 24, 465707. To produce the left-handed and right-handed nanohelices (NHs), the substrate was rotated counterclockwise and clockwise, respectively, at the rotation rate (Rᵣ, in the unit of degree per second) calculated using the following equation,

$${R}_{r} = 360 {R}_{d} / {P}_{NH} \qquad ( 1 )$$

where R_{d} was the calibrated deposition rate of SiO₂ on the substrate surface (0.28 nm s⁻¹) at a of 87° and PNH was the designated pitch of NHs (see Z. Ni, et al., Adv. Sci. 2020, 7, 2001321; and P. Sun, et al., Chem. Commun. 2018, 54, 4270). To produce vertical nanorods, the substrate was rotated at Rᵣ of 6° s⁻¹. The silica NZs deposited on silicon wafers were mechanically spilled, and the freshly exposed cross-sections were characterized with SEM (LEO 1530, Zeiss).

### Animals:

The C57BL/6 mice were purchased from the Laboratory Animal Services Centre of the Chinese University of Hong Kong. The mice were housed in the animal room with a 12 h/12 h light-dark cycle with food and water ad libitum. All experimental procedures on mice were approved by the Department of Health of the HKSAR government.

Primary mBMDC Culture:

The culture media and supplements were purchased from ThermoFisher unless otherwise specified. The culture method of mBMDCs was optimized based on the protocol as described in J. Helft, et al., Immunity 2015, 42, 1197, and M. P. Matheu, et al., J Vis Exp 2008, 17, 773. In particular, the bone marrows were isolated from the femur and tibia of the 6- to 9-week-old C57BL/6 mice. The red blood cells in the bone marrow were lysed by applying a brief hypotonic shock with the autoclaved MilliQ water, which were quenched by adding the corresponding volume of 10×PBS. The isolated mononuclear cell suspension was seeded at a density of 1× 10⁶ cells per mL (3 mL per well on the 6-well plates) in the complete RPMI medium (RPMI 1640 medium supplemented with 15 mm HEPES and 0.5% (v/v) penicillin-streptomycinneomycin (PSN) mixture) supplemented with 10% (v/v) heat-inactivated fetal bovine serum (HI-FBS), and 20 ng mL⁻¹ recombinant murine granulocyte-macrophage colony-stimulating factor (rmGM-CSF, PeproTech, Cat #315-03) and interleukin-4 (rmIL-4, PeproTech, Cat #214-14). On day 3, half of the culture media were replaced with the complete mBMDC medium containing rmGM-CSF and rmIL-4. On day 6, the non-adherent and semi-adherent immature mBMDCs (im-mBMDCs) were collected.

For the mBMDC maturation, the im-mBMDCs were seeded on TCPS plates with or without 100 ng mL⁻¹ LPS (ThermoFisher, Cat #00-4976-93), or seeded on the nanomatrices in the complete RPMI medium supplemented with 10% (v/v) HI-FBS for 20 h. The im-mBMDCs were seeded at a density of 0.7×10⁵ cells per well on 48-well plates with or without 1 cm-diameter nanomatrices for the co-culture with CTLs, 1.2×10⁵ cells per well on 12-mm-diameter polystyrene coverslips (SPL) or 1-cm-diameter nanomatrices on 4-well plates for confocal imaging, 3.5-7×10⁵ cells per well on 12-well plates with or without 2-cm-diameter nanomatrices for flow cytometry and Western blot, and 25×10⁵ cells per well on 6-well plates with or without 3-cm-diameter nanomatrices for in vivo mBMDC injection. To examine the role of FAK in the NZ-mediated effects, the mBMDCs were treated with 5 μm FAK inhibitor PF-573228 (Selleckchem, Cat #S2013) or 0.02% (v/v) DMSO (Sigma, Cat #D2650) at 1 h after cell seeding. To pulse the mBMDCs with antigen OVA₂₅₇₋₂₆₄, the mBMDCs were pre-incubated in the complete RPMI medium supplemented with 1% (v/v) HI-FBS for 30 min, and subsequently treated with 1 nm OVA₂₅₇₋₂₆₄ peptide (InvivoGen, Cat #vac-sin) in the complete RPMI medium supplemented with 1% (v/v) HI-FBS for 2 h. Afterward, the OVA₂₅₇₋₂₆₄ peptide was removed. The antigen-pulsed mBMDCs were then used for co-culture with the CTLs or were detached via the 15-min incubation in an Accutase cell detachment medium (ThermoFisher, Cat #00-4555-56) at 37° C. for the in vivo injection.

Co-Culture of mBMDCs and CTLs:

The 6- to 8-week-old C57BL/6 mice were immunized via the subcutaneous injection of 100 μL mixture of 10 μg OVA₂₅₇₋₂₆₄ peptide and 50% (v/v) complete Freud's adjuvant (InvivoGen, Cat #vac-cfa-10) at the scruff. After 7 days of immunization, the spleen and major lymph nodes were isolated from the immunized mice and dissociated mechanically by smashing the tissues on the cell strainer using the syringe plunger. The red blood cells in splenic cell suspension were lysed via a brief hypotonic shock with sterile MilliQ water which was quenched by adding the corresponding volume of 10×PBS. The CD8⁺ CTLs were purified from the splenic and lymph node cell suspension via the negative cell isolation using the CD8a⁺ T cell isolation kit (Miltenyi Biotec, Cat #130-104-075) according to the manufacturer's instruction. The CTLs were then co-cultured with the OVA₂₅₇₋₂₆₄-pulsed or unpulsed mBMDCs in the complete RPMI medium supplemented with 5% (v/v) HI-FBS in a DC:CTL ratio of 1:10.

Primary hu-moDC Culture:

The peripheral blood mononuclear cells (PBMCs) were isolated from the buffy coat of the healthy donors (obtained from and approved by the Hong Kong Red Cross Blood Transfusion Service; REC reference number: HASC/17-18/0795) using the density gradient centrifuge (Lymphoprep, Stemcell Technologies, Cat #07851). The monocytes were isolated from the PBMCs via positive cell isolation using the human CD14 microbeads (Miltenyi Biotec, Cat #130-050-201) according to the manufacturer's instruction, and seeded at a density of 1.4×10⁶ cells per mL on 6-well plates in serum-free AIM-V medium supplemented with 25 ng mL⁻¹ recombinant human GM-CSF and IL-4 (Miltenyi Biotec, Cat #130-093-866 and 130-093-922). After 24 h of culture, the monocytes were differentiated into the immature hu-moDCs, in which at least 80% of cells had lost the surface expression of CD14. For the maturation of humoDCs, the immature hu-moDCs were seeded in fresh AIM-V medium at a density of 0.6×10⁵ cells per well on 48-well plates with or without the 1-cm-diameter NZs for the co-culture with CTLs, or 2×10⁵ cells per well on 12-well plates with or without 2-cm-diameter NZs for flow cytometry. The hu-moDCs were pulsed with 4 μg mL⁻¹ PepMix human CEA peptide pool (JPT Peptide Technologies, Cat #PM-CEA) right after being seeded on the 12-well or 24-well plates. After 20 h of maturation, the hu-moDCs were used for the subsequent experiments.

Murine Tumor Models and In Vivo mBMDC Injection:

The murine B16 melanoma cells expressing the membrane-bound OVA (B16-OVA, Cat #CE20513, Crisprbio, Beijing, China) were cultured in the DMEM supplemented with high glucose, GlutaMAX, 10% (v/v) HI-FBS, 0.5% (v/v) PSN and 400 μg mL⁻¹ selective agent G418 sulfate (ThermoFisher, Cat #10131035). To ensure that most of the B16-OVA cells could enter the exponential growth phase at the time of injection, the cells used for in vivo inoculation had passage number≤15 and were expanded in the complete DMEM medium without selective agent G418. The B16-OVA cells were detached using the TrypLE Express enzyme (ThermoFisher, Cat #12604021) and passed through the 100-μm cell strainer. The 9- to 11-weekold C57BL/6 female mice (with an average weight of 20 g) were subcutaneously injected with 2.5×10⁵ B16-OVA cells in 50 μL HBSS into their shaved right flanks, under ketamine-xylazine anesthesia. On day 8 after the inoculation of cancer cells, the visible tumors were formed. The tumor-bearing mice were randomly divided into three groups. Under ketamine xylazine anesthesia, 1.75×10⁶ OVA₂₅₇₋₂₆₄-pulsed mBMDCs maturated with silica NZs or LPS in 100 μL HBSS were subcutaneously injected into the tumor-bearing mice at the area next to the tumor. The sham control group was subcutaneously injected with 100 μL HBSS without any mBMDCs. Tumor size was monitored every two days using the calipers for a total of 14 days. The tumor volume was calculated using the following formula:

$$( Length \times {Width}^{2} ) / 2 \qquad ( 2 )$$

The mice were sacrificed and considered as death when their tumor volume reached 1000 mm³.

### Statistical Analysis:

The statistical analyses were conducted in the Prism 9 software (GraphPad), except that the homogeneity of variance between groups was analyzed using Levene's test based on median in the SPSS Statistics version 26. According to the Levene's test results, two-tailed Student's t-test or two-tailed Welch's t-test was used for the mean comparison between two groups; and one-way ANOVA followed by Tukey's HSD post-hoc test or Welch's ANOVA followed with Dunnett's T3 post-hoc test was used for the mean comparison of more than two groups. Two-way ANOVA followed by Tukey's HSD post-hoc test or Šídák's multiple comparisons test was used for the mean comparison between groups with two independent variables. The p-values <0.05 were considered as statistically significant. Numerical data were presented as mean±standard error of the mean.

Scanning Electron Microscopy on the mBMDCs Maturated with Silica NZs:

The mBMDCs were seeded on the silica NZs deposited on the silicon wafer. At 20 hours after cell seeding, the cells were fixed with 4% (w/v) paraformaldehyde (PFA) in warm culture medium for 2 hours at 37° C. and post-fixed in 4% (w/v) PFA in PBS for two days at 4° C. The fixed cells were dehydrated with a series of gradient ethanol, and then chemically dried by immersing in the hexamethyldisilazane (Electron Microscopy Sciences, Cat #16700) for 3 minutes at room temperature. The dried samples were gold-sputtered and visualized under the SEM (LEO 1530, Zeiss).

### Co-Culture of Hu-moDCs and Autologous CTLs:

The autologous PBMCs were frozen in the freezing medium (70% (v/v) AIM-V+20% (v/v) HI-FBS+10% (v/v) DMSO) and kept in liquid nitrogen. On the day of co-culture, the CD8⁺ CTLs were isolated from the thawed PBMCs via negative cell isolation using the human CD8⁺ T cell isolation kit (Miltenyi Biotec, Cat #130-096-495) according to the manufacturer's instruction. To yield the naïve CD8⁺ CTLs, the terminally differentiated CTLs were further depleted from the CTL suspension using the human CD57 microbeads (Miltenyi Biotec, Cat #130-092-073). The CD8+CD57-naïve CTLs were then co-cultured with the hu-moDCs in the AIM-V medium supplemented with 5% (v/v) heat-inactivated human AB serum (Sigma, Cat #H5667) in the DC:CTL ratio of 1:5.

### Flow Cytometry on Dendritic Cells:

The adherent mBMDCs or hu-moDCs were detached by incubating in the Accutase cell detachment medium for 15 minutes at 37° C. For surface protein staining, the collected cells were incubated with the mixture of primary antibodies (Table 1) and fixable viability dye eFluor 780 (FVD780, ThermoFisher, Cat #65-0865-14) diluted in the FACS buffer (PBS+2% (v/v) HI-FBS) for 30 minutes at 4° C. For intracellular cytokine staining, the adherent mBMDCs were treated with 0.1% (v/v) GolgiPlug protein transport inhibitor (BD Bioscience, Cat #555029) in complete RPMI medium supplemented with 5% (v/v) HI-FBS for 16 hours before harvest. After staining for the surface proteins, the cells were fixed and permeabilized using the Foxp3/Transcription Factor Staining Buffer Set (ThermoFisher, Cat #00-5523-00), and then incubated with the anti-cytokine primary antibodies (Table 1) diluted in 1× permeabilization buffer for 2 hours at 4° C. The stained cells were washed and suspended in the FACS buffer for flow cytometry (FACSCanto II, BD Bioscience). The flow cytometry results were analyzed in the FlowJo 10.4 software (BD Bioscience). For all analyses, the cell debris and cell doublets were gated out using the FSC-A vs. SSC-A plot and FSC-A vs. FSC-H plot, respectively. The dead cells were gated out using the FVD780 staining results. The CD11c⁺ MHC-II⁺ cells were defined as mBMDCs and hu-moDCs.

TABLE 1
List of antibodies and tetramers used

| | | | | |
|---|---|---|---|---|
| Target protein/ | | | | |
| Chemical name | Conjugate | Clone | Cat# | Manufacturer |

Anti-mouse primary antibodies for flow cytometry

| | | | | |
|---|---|---|---|---|
| CCR7 | BV421 | 4B12 | 566291 | BD Bioscience |
| CD11c | BV510 | N418 | 744178 | BD Bioscience |
| CD206 | PE/Cy7 | C068C2 | 141719 | BioLegend |
| CD3 | BV510 | 17A2 | 100234 | BioLegend |
| CD44 | PE/Cy7 | IM7 | 103029 | BioLegend |
| CD44 | BB700 | IM7 | 566507 | BD Bioscience |
| CD80 | PE-Cy7 | 16-10A1 | 104734 | BioLegend |
| CD8a | AF488 | 53-6.7 | 100723 | BioLegend |
| CD8a | PE/Cy7 | 53-6.7 | 100722 | BioLegend |
| DC-SIGN | BV421 | 5H10 | 747827 | BD Bioscience |
| H-2K^{b}/H-2D^{b} | PE | 28-8-6 | 114607 | BioLegend |
| H-2k^{b}-OVA₂₅₇₋₂₆₄ | PE | eBio25-D1.16 | 12-5743-81 | ThermoFisher |
| I-A/I-E | AF488 | M5/114.15.2 | 107616 | BioLegend |
| IFN-γ | AF647 | XMG1.2 | 505816 | BioLegend |
| IL12-p40 | PE | C15.6 | 505203 | BioLegend |
| PD-1 | PE/Cy7 | 29F.1A12 | 135215 | BioLegend |
| PD-1 | APC | 29F.1A12 | 135209 | BioLegend |
| T-bet | PE | eBio4B10 (4B10) | 12-5825-80 | ThermoFisher |
| TNFα | AF647 | MP6-XT22 | 506314 | BioLegend |
| XCR1 | AF647 | ZET | 148214 | BioLegend |

Anti-mouse H-2k^{b}-OVA₂₅₇₋₂₆₄ tetramers for flow cytometry

| | | | | |
|---|---|---|---|---|
| Flex-T Biotin H-2K(b) | N/A | N/A | 280051 | BioLegend |
| OVA Monomer | | | | |
| (SIINFEKL) | | | | |
| Streptavidin | BV421 | N/A | 405226 | BioLegend |
| D-Biotin | N/A | N/A | B20656 | ThermoFisher |
| Sodium Azide | N/A | N/A | S2002 | Sigma |

Anti-human primary antibodies for flow cytometry

| | | | | |
|---|---|---|---|---|
| CCR7 | PE | 2-L1-A | 566742 | BD Bioscience |
| CD11c | BV510 | B-ly6 | 563026 | BD Bioscience |
| CD80 | PE/Cy7 | L307.4 | 561135 | BD Bioscience |
| CD86 | BV421 | 2331 (FUN-1) | 562432 | BD Bioscience |
| CD107a | BB700 | H4A3 | 566558 | BD Bioscience |
| DC-SIGN | BB515 | DCN46 | 564548 | BD Bioscience |
| HLA-ABC | PE | G46-2.6 | 555553 | BD Bioscience |
| HLA-DR | APC | G46-6 | 559866 | BD Bioscience |
| IFNγ | AF647 | B27 | 557729 | BD Bioscience |
| XCR1 | BV421 | S15046E | 372609 | BioLegend |

Primary antibodies for Western blot and immunofluorescence

| | | | | |
|---|---|---|---|---|
| GAPDH | HRP | D16H11 | 8884S | Cell Signaling |
| | | | | Technology |
| FAK | None | 77/FAK | 610087 | BD Bioscience |
| Phospho-FAK (Tyr397) | None | D20B1 | 8556S | Cell Signaling |
| | | | | Technology |
| Vinculin | None | hVIN-1 | V9264 | Sigma |
| PIP2 | None | 2C11 | ab11039 | Abcam |

Secondary antibodies for Western blot and immunofluorescence

| | | | | |
|---|---|---|---|---|
| Anti-Mouse IgG(H + L), | AF594 | N/A | A11020 | ThermoFisher |
| F(ab′)2 fragment | | | | |
| Anti-Mouse IgG(H + L), | AF647 | N/A | 115-607-003 | Jackson |
| F(ab) fragment | | | | ImmunoResearch |
| | | | | Labs |
| Anti-Mouse IgG | HRP | N/A | 7076 | Cell Signaling |
| | | | | Technology |
| Anti-Rabbit IgG(H + L), | AF647 | N/A | A21246 | ThermoFisher |
| F(ab′)2 fragment | | | | |
| Anti-Rabbit IgG | HRP | N/A | 7074 | Cell Signaling |
| | | | | Technology |

### Flow Cytometry on CTLs:

The suspended murine and human CTLs were harvested for flow cytometry. To stain for the OVA₂₅₇₋₂₆₄-specific murine CTLs, the BV421-conjugated H-2k^{b}-OVA₂₅₇₋₂₆₄ tetramers were prepared and used as instructed by the manufacturer (BioLegend). The chemicals for preparing the tetramers and the primary antibodies for staining CTLs are listed in Table 1. The surface proteins of CTLs were stained in the same practice as the DCs mentioned above. For the intracellular cytokine staining, the CTLs were treated with 0.1% (v/v) GolgiPlug for 6 hours before harvest. After staining for the surface proteins, the cells were fixed and permeabilized using the CytoFix/CytoPerm Kit (BD Bioscience, Cat #554714), and then incubated with the anti-cytokine primary antibodies (Table 1) diluted in 1× Perm/Wash buffer overnight at 4° C. The CTLs treated with 1× eBioscience cell stimulation cocktail (ThermoFisher, Cat #00-4970-93) were served as the positive control for IFNγ expression. For the staining of transcription factors, after staining for the surface proteins, the cells were fixed and permeabilized using the Foxp3/Transcription Factor Staining Buffer Set, and then incubated with the anti-transcription factor primary antibodies (Table 1) diluted in 1× permeabilization buffer overnight at 4° C. For the CFSE-based T-cell proliferation assay, the murine CTLs were pre-stained with 1 μm CFSE (ThermoFisher, Cat #C34554) in HBSS supplemented with 15 mM HEPES and 2 mm EDTA for 5 minutes at room temperature. The unbound dyes were absorbed by adding complete RPMI medium supplemented with 5% (v/v) HI-FBS. The stained CTLs were then centrifuged and resuspended in complete RPMI medium supplemented with 5% (v/v) HI-FBS for the mBMDC-CTL co-culture. The CTLs treated with 2.5 μg mL⁻¹ phytohemagglutinin-L (ThermoFisher, Cat #00-4977-93) were served as the positive control for CFSE dilution. The stained cells were suspended in the FACS buffer for flow cytometry (FACSCanto II, BD Bioscience). The flow cytometry results were analyzed in the FlowJo 10.4 software (BD Bioscience). For all analyses, the cell debris were gated out using the FSC-A vs. SSC-A plot and the dead cells were gated out using the FVD780 staining results.

### Fluorescence Staining and Confocal Imaging:

The silica NZs were pre-stained with 1 UM CellTracker Deep Red dye (ThermoFisher, Cat #C34565) in PBS for 30 minutes at 37° C. The unbound dye was washed with PBS and absorbed by the RPMI medium with 10% (v/v) HI-FBS for at least 1 hour at 37° C. before seeding the mBMDCs on the CellTracker Deep Red dye-stained silica NZs. After being seeded on the polystyrene coverslips or silica NZs for 20 hours, the mBMDCs were fixed with 2% (w/v) PFA for 30 minutes at 37° C. and post-fixed with warm 4% (w/v) PFA for 10 minutes at room temperature. The free aldehyde groups on the fixed cells were blocked by incubating the cells in 0.2% (w/v) glycine in PBS for 30 minutes at room temperature. To visualize the cell membrane, the fixed cells were stained with 1 μg mL⁻¹ CF488A-conjugated Wheat Germ Agglutinin (WGA, Biotium, Cat #29022) for 30 minutes at room temperature. For the immunostaining of vinculin, p-FAK (Y397) and PIP2, the fixed cells were permeabilized and blocked with 0.1% (v/v) Triton X-100 and 5% (w/v) BSA in PBS for 30 minutes at room temperature. The cells were then incubated in the mixture of primary antibodies (Table 1) diluted in PBS supplemented with 1% (v/v) normal goat serum, 5% (w/v) BSA and 0.01% (v/v) Triton X-100 overnight at 4° C. On the next day, the cells were washed and incubated in the mixture of Alexa Fluor 594-conjugated F(ab′)² fragment anti-mouse IgG antibodies (ThermoFisher, Cat #A11020) and Alexa Fluor 647-conjugated F(ab′) 2 fragment anti-rabbit IgG antibodies (ThermoFisher, Cat #A21246) diluted in PBS supplemented with 0.01% (v/v) Triton X-100 for 2 hours at room temperature. For the double immuno-staining of vinculin and PIP2, of which primary antibodies were raised from mouse, excess Alexa Fluor 647-conjugated monovalent F (ab) anti-mouse secondary antibody (Jackson ImmunoResearch Labs, Cat #115-607-003) was used to tag and saturate the first anti-vinculin primary antibody. After thorough washing, the cells were incubated with the antiPIP2 primary antibody overnight at 4° C., and with the Alexa Fluor 594-conjugated F(ab′)² fragment anti-mouse IgG antibodies for 2 hours at room temperature on the next day. The cell nuclei were stained with 4 μg mL⁻¹ Hoechst 33342 (ThermoFisher, Cat #H3570) for 5 minutes at room temperature. The F-actin in cells were stained with 0.0125% (v/v) Alexa Fluor 488 Phalloidin (ThermoFisher, Cat #A12379) for 1 hour at room temperature. The stained cells were then mounted in the ProLong Glass antifade mounting medium (ThermoFisher, Cat #P36980) on the #1.5H rectangular coverslips (Marienfeld GmbH), and then healed in dark for at least 4 days before visualized under the microscope.

The confocal laser scanning microscope Stellaris 5 (Leica) equipped with 405 nm, 488 nm, 561 nm and 638 nm laser and installed with LAS X software was used for capturing the confocal images. For the visualization of the cell membranes of mBMDCs and the cell adhesions formed at the DC-NZ interfaces, the stained cells were captured as multi-channel z-stacks under the 63× oil immersion lens (NA: 1.4) of confocal microscope with the acquisition parameters set as 16-bit, 1 airy unit pinhole size, 60 nm pixel size and 130 nm z-step. The images with fluorescence signals of CellTracker Deep Red dye-stained NZs or vinculin were first background subtracted with 30-pixel rolling ball radius in the Fiji software. The images with signals of NZs, WGA-stained membrane, vinculin or F-actin were then deconvoluted using the theoretical point spread function and the Classic Maximum Likelihood Estimation method in the Huygens Essential software (version 17.10.0p2, Scientific Volume Imaging).

Image Acquisition and Quantification for Cellular Adhesions and p-FAK Level:

The fluorescence signals of F-actin, vinculin and p-FAK at the cell membrane proximal to the polystyrene coverslips or silica NZs were captured as multi-channel Z-stacks under the 63× oil immersion lens (NA: 1.4) of the Stellaris 5 confocal microscope (Leica) with the acquisition parameters set as 16-bit, 1 airy unit pinhole size, 90 nm pixel size and 300 nm z-step. The samples were randomly coded and not identified until all samples within one trial had been analyzed. At least 50-70 cells were captured for each group per trial. The captured images were analyzed using the macros and batch processing in the Fiji software. In brief, the images with vinculin or p-FAK signals were background subtracted with 10-pixel rolling ball radius. The maximum intensity projected (MIP) images with F-actin signal were applied with Gaussian Blur filter (sigma=1) and segmented using the “Triangle” auto-threshold method followed by a succession of “close” and “fill holes” operations. The cell spreading area was measured as the area of F-actin mask. The background vinculin and p-FAK signals outside of the dilated F-actin mask were removed by the “clear outside” command. To create the 3D mask for vinculin+adhesions, the image stacks with vinculin signals were applied with top hat filter (radius=4 pixels) and Gaussian Blur filter (sigma=1). Except for the cells with adhesions covering over 50% of cell area, the cytoplasmic vinculin signals were reduced by subtracting from the mean grey value of the region of interest (ROI) at the cytoplasmic region without adhesions. The vinculin+adhesion masks were generated using the “IJ_IsoData” auto-threshold method, and converted to the merged 3D ROI via the “Analyze Particles” and “3D Manager” plug-ins. The integrated density (IntDen) of p-FAK signals within the 3D ROI of vinculin+adhesion mask was measured in the “3D Manager” plug-in. The fluorescence intensity of p-FAK in vinculin+adhesions in each cell was quantified by dividing the IntDen of p-FAK signals in the 3D ROI by the volume of 3D ROI. To measure the total area covered with vinculin+adhesions, the stack of vinculin⁺ adhesion mask was sum-projected and converted into a new mask using the “Triangle” auto-threshold method. The percentage of cell area covered with vinculin⁺ adhesions were calculated by (area of sum-projected adhesion mask-area of un-dilated F-actin mask)×100%.

Uptake Assay of Dextran and Microspheres in mBMDCs:

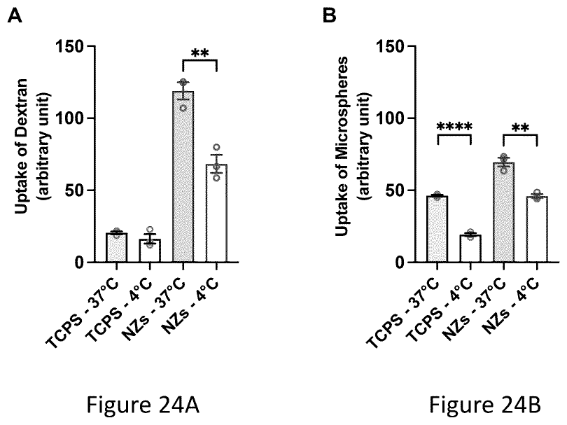

The mBMDCs seeded on polystyrene coverslips or silica NZs were pre-incubated in complete RPMI medium supplemented with 1% (v/v) HI-FBS for 30 minutes, and then treated with 0.5 mg mL⁻¹ AF647-conjugated dextran (average molecular weight of 10000, ThermoFisher, Cat #D22914) or 0.01% (v/v) dark red fluorescent microspheres (40-nm diameter, ThermoFisher, Cat #F8789) in staining buffer (HBSS supplemented with 0.5 mm magnesium chloride, 0.9 mm calcium chloride and 15 mm HEPES) for 15 minutes at 37° C. To ensure that the increase of intracellular fluorescence was led by the endocytosis, the cells treated with dextran or microspheres for 15 minutes at low temperature (4° C.) were served as negative control (FIGS. 24A to 24B). Afterward, the fluorescence signals of the extracellular dextran and microsphere were quenched by incubating the cells in 0.25 mg mL⁻¹ Trypan blue diluted in staining buffer for 1 minute at room temperature. The cells were then washed and fixed with 4% (w/v) PFA for 15 minutes at room temperature, and then permeabilized with 0.1% (v/v) Triton X-100 for 10 minutes at room temperature. The fixed cells were counterstained for nuclei and F-actin, and mounted on #1.5H coverslips as mentioned above. The stained cells were captured as multi-channel images under the 63× oil immersion lens (NA: 1.4) of the Stellaris 5 confocal microscope (Leica) with the acquisition parameters set as 8-bit, 1 airy unit pinhole size and 180 nm pixel size. The samples were randomly coded and not identified until all samples within one trial had been analyzed. At least 200 cells from a minimum of 30 images were captured for each group per trial. With a total of five independent trials, at least 1000-1300 cells were measured for each group. The captured images were analyzed using the macros and batch processing in the Fiji software. In brief, the images with dextran or microsphere signals were background subtracted with 10-pixel rolling ball radius. To visualize the cell boundaries, the images with F-actin signals were segmented using the “Triangle” auto-threshold method. The raw IntDen of dextran or microsphere signals within the F-actin mask were measured using the ROI manager. The intracellular fluorescence intensity of dextran or microsphere was calculated by dividing the raw IntDen of dextran or microsphere signals by the area of F-actin mask.

### Western Blotting:

The mBMDCs were detached using the cell scrapper and lysed in the radioimmunoprecipitation assay (RIPA) buffer (Millipore) containing 1% (v/v) Halt Protease inhibitor cocktail (ThermoFisher), 1% (v/v) phosphatase inhibitor mixture 2 and 1 mm phenylmethylsulfonyl fluoride (Sigma). The cell lysates were agitated for 90 minutes at 4° C. and centrifuge at 14000 rcf for 15 minutes at 4° C. The amount of extracted protein in the supernatant was examined using the Quick Start Bradford Protein Assay (Bio-rad). The protein extracts were boiled in 4× Laemmli buffer with 10% (v/v) B-mercaptoethanol for 5 minutes at 99° C., separated by the 7.5% SDS-PAGE gel, and transferred onto the polyvinylidene difluoride membrane (Millipore). The membranes were blocked with 5% (w/v) non-fat milk in TBST, and incubated with the primary antibodies (Table 1) diluted in 5% (w/v) BSA in TBST overnight at 4° C. The probed membranes were washed and incubated with the HRP-conjugated goat anti-rabbit IgG or anti-mouse IgG secondary antibodies (Cell Signaling Technology) diluted in 2.5% (w/v) non-fat milk in TBST for 1 hour at room temperature. The HRP signals were detected using the Immobilon ECL Ultra Western HRP Substrate (Millipore), captured using the ChemiDoc Imaging System (Bio-rad) and quantified using the Fiji Software. After probing for the p-FAK, the membranes were stripped by washing with mild stripping buffer (200 mm glycine, 0.1% (w/v) SDS, and 1% (v/v) Tween 20, pH 2.2) for 10 minutes twice at room temperature, before re-probing for the total FAK.

### Example 2

Effects of the Silica Nanostructures on the Maturation of mBMDCs

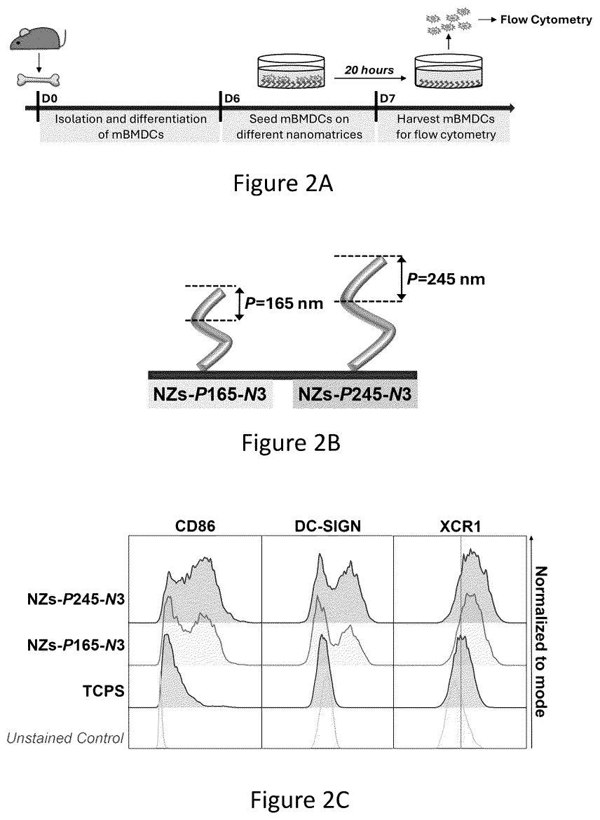

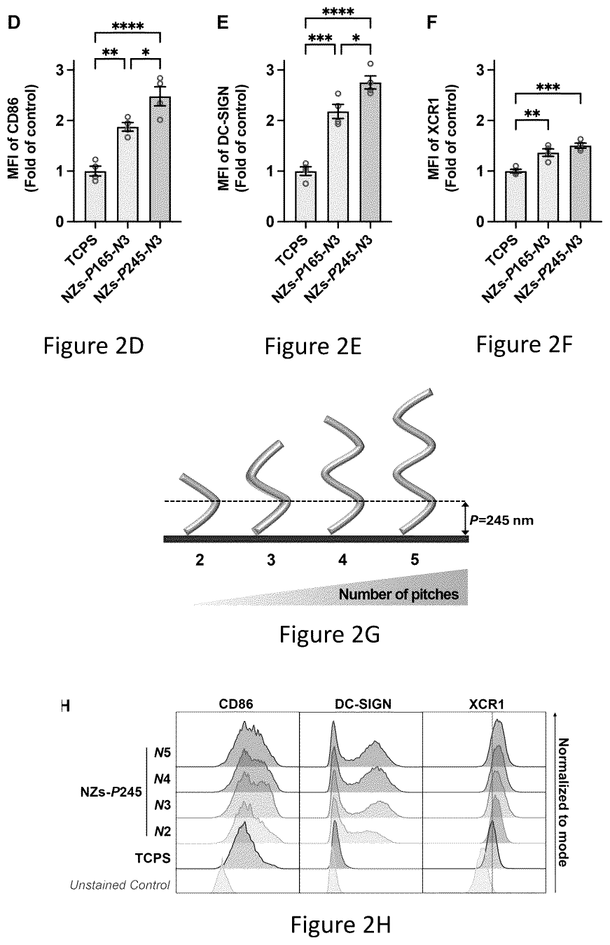

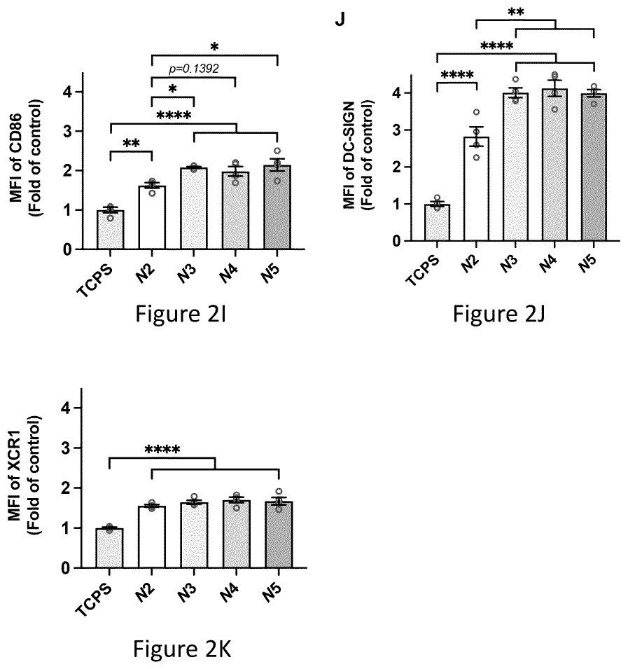

The mBMDCs were applied to the silica nanomatrices for 20 h to induce their maturation, in the absence of cytokines or maturation chemicals (FIG. 2A). The immature mBMDCs adhered to tissue culture polystyrene (TCPS) without any maturation treatment were served as negative control group. To optimize the silica nanomatrix-mediated maturation of mBMDCs, the shapes (nanohelices or NHs with left and right handedness, vertical nanopillars or vNPs, tilted nanopillars or tNPs, and NZs) and nanostructures (including P and N) of silica nanomatrices were tuned by GLAD. For DCs, CD86 is an important co-stimulatory molecule for activation of T cells, of which expression level is well-reported to be upregulated upon the maturation of DCs. The DC-specific ICAM-3 grabbing nonintegrin (DC-SIGN) is a C-type leptin receptor (CLR) on the DCs to facilitate the uptake of antigens and the initial contact between DCs and T cells for the priming of T cells. The X-C motif chemokine receptor-1 (XCR1) is a receptor for the X-C motif chemokine ligand-1 (XCL1) released by the activated CTLs, of which expression on the DCs could sustain the interaction between DCs and CTLs and augment the CTL-mediated response against cancer cells. Therefore, the upregulated expressions of these proteins on the maturated DCs indicate the increased capacity of DCs in potentiating the anti-tumor CTLs, and thus, were picked as the markers to choose the optimal silica nanomatrices for in vitro maturation of DCs.

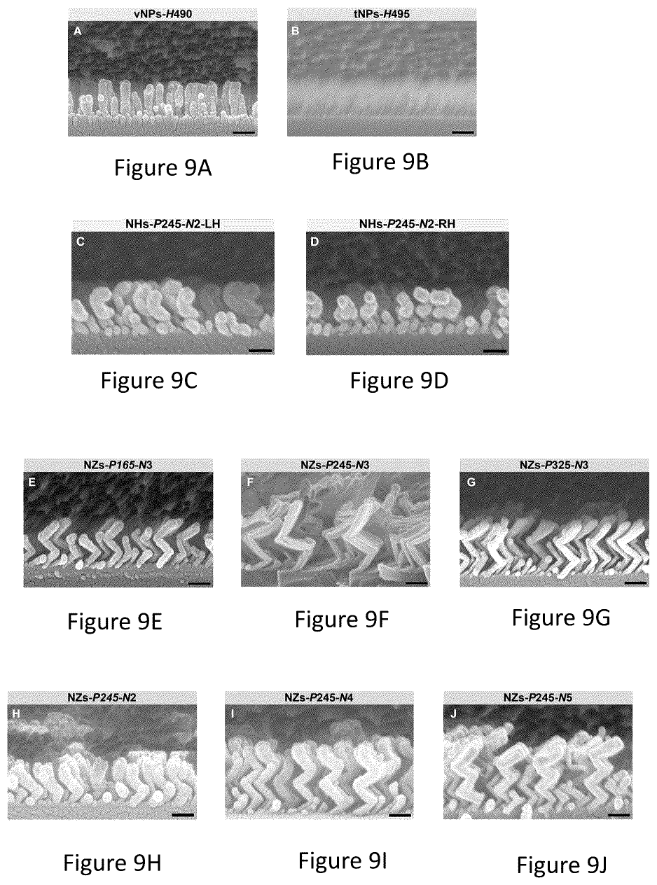

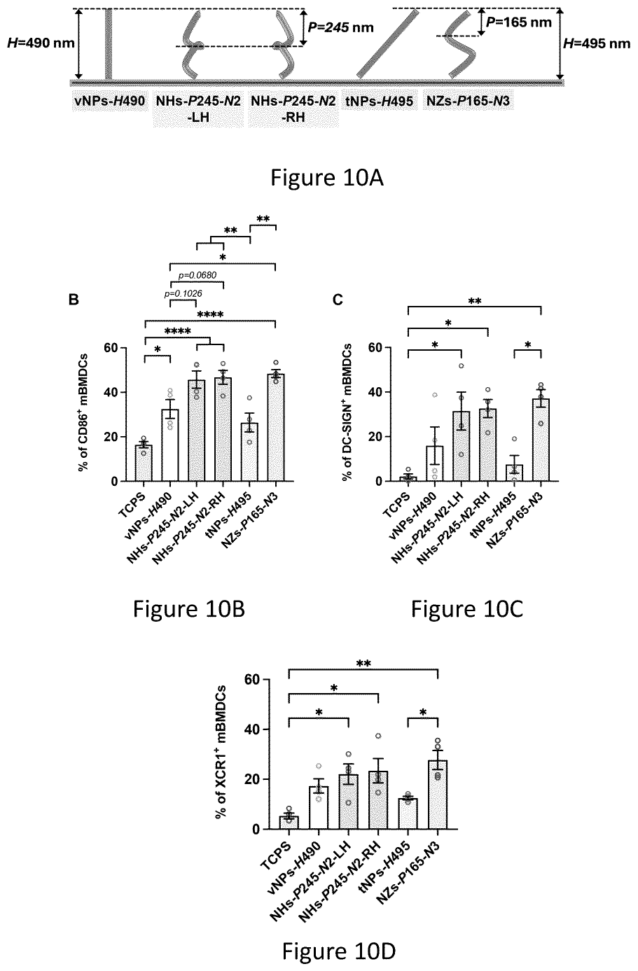

First, to study the effect of the shape of silica nanomatrices, the mBMDCs were maturated on the silica nanomatrices (with a height H of 490-495 nm) sculpted in a shape of vNPs (i.e., vNPsH490), left-handed and right-handed NHs (with a P of 245 nm and N of 2, i.e., NHs-P245-N2-LH and NHs-P245-N2-RH), tNPs (i.e., tNPs-H495) and NZs (with a P of 165 nm and N of 3, i.e., NZs-P165-N3) (FIGS. 9A-E and 10A). All the silica nanomatrices, except for the tNPs, significantly increased the percentage of CD86⁺ mBMDCs, wherein the highest increase was induced by the NHs and NZs (FIG. 10B). The NHs and NZs, instead of vNPs and tNPs, enhanced the percentage of DC-SIGN⁺ and XCR1⁺mBMDCs (FIGS. 10C and 10D). The handedness of NHs showed a negligible effect (FIGS. 10B to 10D). Thus, the effect of the nano-shape on the upregulation of these proteins could be summarized as an ascending order of tNPs<vNPs<NHs≈NZs. As demonstrated by the data distribution and the higher statistical significance in the pair-wise comparison with the TCPS control group, the NZs tended to result in lower variation in the increase of DC-SIGN+ and XCR1+ mBMDCs (FIGS. 10C and 10D). This observation suggests that, compared with the NHs, the NZs could induce a more consistent enhancement in the expressions of DC-SIGN and XCR1 in mBMDCs, implying that the shape of NZ has an advantage in the in vitro maturation of mBMDCs.

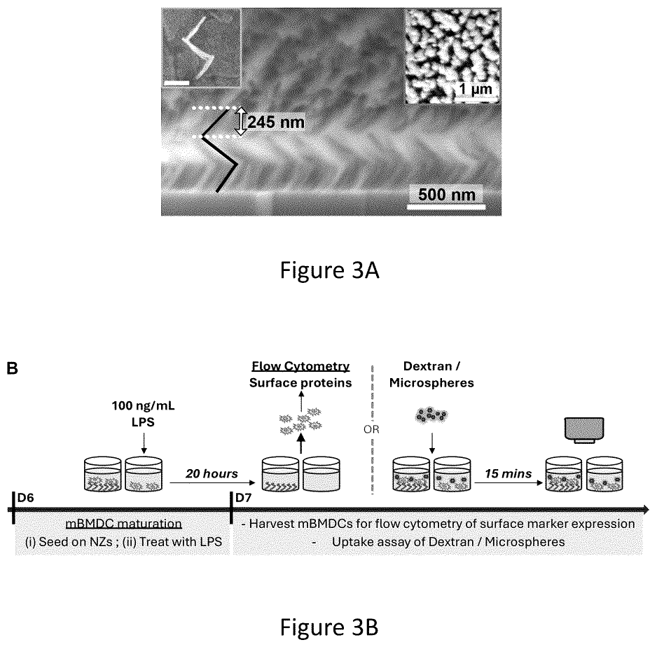

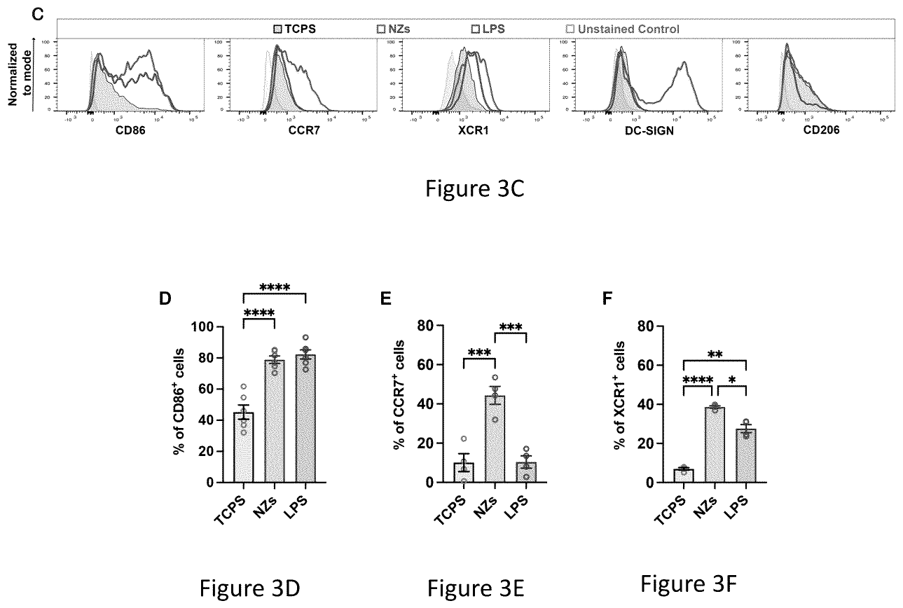

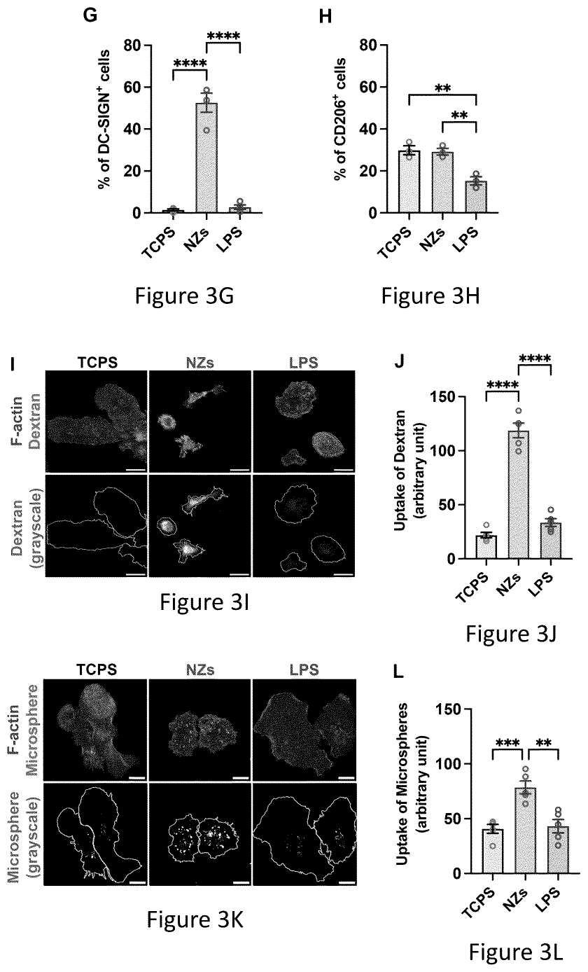

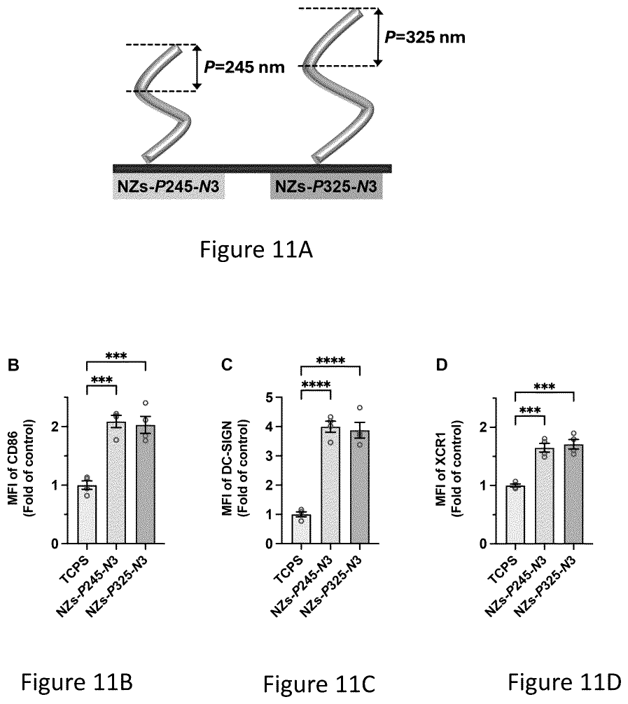

Next, the NZ-induced maturation of mBMDCs were further optimized by engineering the nanostructures of silica NZs, in terms of P and N. To study the effect of P, P was raised from 165 nm to 245 nm (i.e., NZs-P165-N3 versus NZs-P245-N3; FIG. 2B; FIGS. 9E to 9F) at a given N of 3. The results showed that the NZs-P245-N3 led to further enhancement in the expression levels of CD86 and DC-SIGN, while no significant improvement was observed in the expression of XCR1 (FIG. 2C-F). As Young's modulus of silica NZs is reported to increase with the rise of P, these findings demonstrate that the stiffness of silica NZs possibly plays a role in modulating the NZ-induced expressions of CD86 and DC-SIGN, but not XCR1, in mBMDCs. An additional increase of P to 325 nm had no further effect on the upregulation of CD86, DC-SIGN, and XCR1 (FIGS. 9F, 9G and 11A to 11D). These findings indicate that larger P is more favorable for the maturation of mBMDCs, while P>245 nm will not promote the maturation to a greater extent. Then, the effect of N in a range of 2-5 on the NZ-induced maturation of mBMDCs was studied at the P of 245 nm (FIG. 2G; FIGS. 9F, 9H to 9J). All the NZs-P245 with diverse N values led to a significant increase in the expression levels of CD86, DCSIGN, and XCR1, compared with the TCPS control (FIGS. 2H to 2K). Compared with the N of 2, the N of 3-5 induced a notable enhancement in the expression levels of CD86 and DC-SIGN (FIG. 2I-J). No significant difference in the expression levels of CD86 and DC-SIGN was detected among the N of 3-5. These results illustrate that the NZ-induced maturation of mBMDCs is promoted by an increase of N and reaches the optimum at N≥3. Overall, the findings demonstrate that engineering the NZ nanostructures could modulate the expression levels of CD86 and DC-SIGN, but not XCR1, in the mBMDCs. Furthermore, silica NZs with a P of 245 nm and an N of 3 are the optimal nanostructures for the maturation of mBMDCs. Therefore, the silica NZs-P245-N3 (FIG. 3A) was chosen for the subsequent experiments, see Examples 3 to 7.

### Example 3

Characterization of the Silica NZ-Maturated mBMDCs

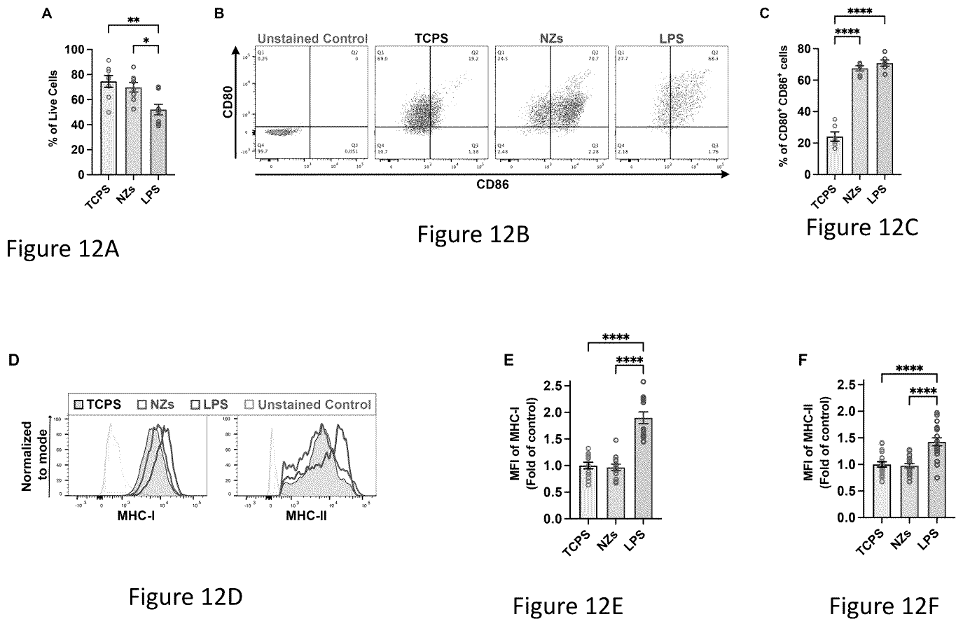

To understand how the silica NZ-induced maturation of mBMDCs differs from the conventional methods, the NZs-P245-N3-maturated mBMDCs were compared with the TCPS-attached mBMDCs maturated via a 20 h treatment of 100 ng mL⁻¹ LPS (FIG. 3B). The silica NZs did not have a cytotoxic effect on the mBMDCs (FIG. 12A). For the expressions of surface proteins associated with the maturation of DCs, both the silica NZs and LPS significantly increased the percentage of mBMDCs expressing the co-stimulatory molecule CD86 to a comparable extent (FIGS. 3C, 3D, 12B and 12C). Compared with the LPS-maturated mBMDCs, the NZ-maturated mBMDCs did not increase the expressions of major histocompatibility complex class I and II (MHC-I and MHCII; FIGS. 12D to 12F) but upregulated the level of lymph-node-homing factor C-C chemokine receptor type-7 (CCR7, FIGS. 3C and 3E). Moreover, for the expressions of XCR1 and DC-SIGN, which were previously found to be upregulated by silica NZs (FIGS. 2E, 2F, 2J, 2K), LPS also increased the expression of XCR1 in the mBMDCs, but to a lesser extent, compared with the silica NZs (FIGS. 3C, 3F). However, LPS did not enhance the expression of DC-SIGN as the silica NZs did (FIGS. 3C, 3G). Besides being an adhesion molecule for promoting the interaction of DCs and T cells, the DC-SIGN is also a CLR for the internalization of exogenous antigens. The increased level of DC-SIGN in the NZ-maturated mBMDCs illustrates the possible enhancement of antigen-uptake capacity. Hence, the inventors further examined the expression of another CLR related to antigen uptake, mannose receptor (CD206), and studied the endocytic capacity of mBMDCs via the uptake assay of dextran and microspheres. The expression level of CD206 was diminished in the LPS-maturated mBMDCs but not in the NZ-maturated mBMDCs (FIG. 3C,H). Consistently with the expression patterns of these CLRs, the significant rise of the uptake of CLR ligand, dextran, was only observed in the NZ-maturated mBMDCs but not in the LPS-maturated ones (FIG. 3I,J). The number of internalized microspheres in the NZ-maturated mBMDCs was also larger than those in the LPS-maturated mBMDCs (FIG. 3K,L), indicating a higher pinocytic or phagocytic activity in the NZ-maturated mBMDCs. These findings demonstrate that silica NZs promote the maturation of mBMDCs and enhance the expressions of CD86, CCR7, XCR1, and DC-SIGN, as well as the endocytic capacity, in the mBMDCs. Compared with the LPS-maturated mBMDCs, the NZ-maturated mBMDCs have a higher level of XCR1, CCR7, DC-SIGN, and endocytic capacity.

To induce a specific antigen-targeted immune response, the in vitro maturated DCs shall first be loaded with antigens. In contrast to the low expression level of CLRs and modest endocytic capacity in the LPS-maturated mBMDCs, the NZ-maturated mBMDCs retained an upregulated expression level of CLRs and high endocytic capacity after the 20 h maturation period. Previous studies have shown that the LPS stimulation could only initiate a few-hour upregulation of antigen uptake at the early phase of maturation. The expression of CLR, CD206, and antigen-uptake capacity were then downregulated in the LPS-maturated DCs, see A. Alloatti, et al., Immunol Rev 2016, 272, 97. These might lead to limited loading of tumor antigens and difficulties in optimizing the timing for antigen loading in the development of the DC vaccine. Therefore, the persistently high endocytic capacity in the NZ-maturated mBMDCs might overcome this problem and result in a more efficient loading of antigens.

Besides, the NZ-maturated mBMDCs express enhanced levels of DC-SIGN which is proposed as the target to facilitate the delivery of antigens into the DCs. The antigens conjugated with the antibodies targeting the extracellular neck domain of DC-SIGN could be internalized into the DCs via the DC-SIGN, and be retained in the early endosome to be loaded into the MHC-I complex for the cross-presentation to CTLs. Thus, it is possible to combine the NZ-induced DC maturation with the DC-SIGN-targeted delivery of tumor antigens to facilitate the generation of maturated DCs with cross-presentation of tumor antigens for the priming of antigen-specific CTLs. After the uptake of antigens, the mBMDCs should present the antigens in the MHC-I complexes that specifically bind to the T-cell receptor (TCR) on the antigen-specific CTLs. Then, the other co-stimulatory signals, in the form of direct interaction with surface proteins or paracrine signaling, are required to stimulate the survival, proliferation, and production of cytokines in the antigen-specific CTLs. Although not have a significant increase in the expression of MHC-I, the NZ-maturated mBMDCs expressed the baseline level of MHC-I for presenting the antigens to the CTLs.

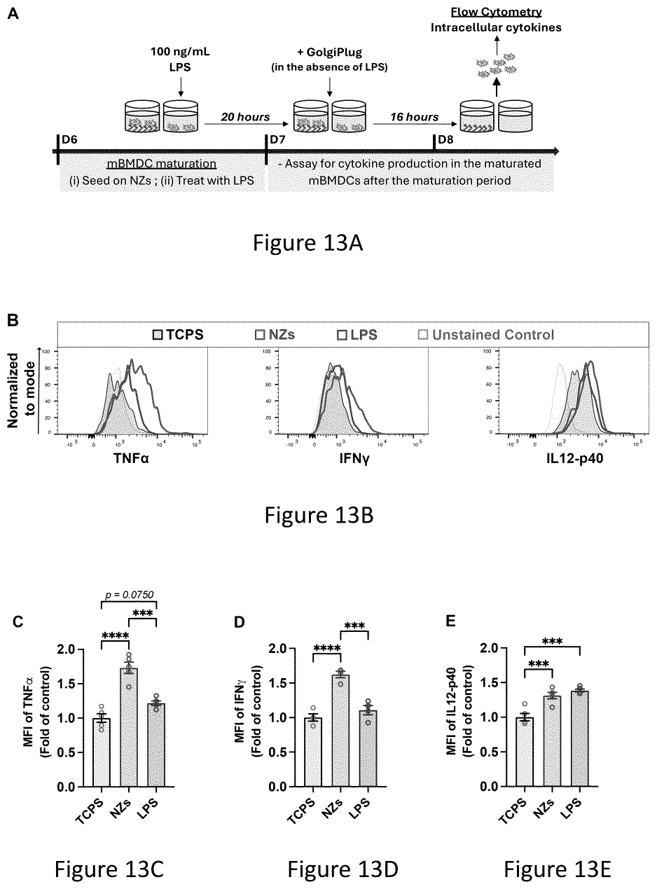

Both the silica NZs and LPS resulted in a distinctive increase of mBMDCs expressing the co-stimulatory molecules, CD86, which could bind to the CD28 receptors on the CTLs to provide the crucial co-stimulatory signals for the activation of CTLs. Therefore, both the NZ-maturated and LPS-maturated mBMDCs should be capable of priming the CTLs. Nevertheless, DC-SIGN is a high-affinity receptor for the intercellular adhesion molecule-3 expressed on the resting T cells, which promotes the initial contact of mBMDCs with the CTLs. Besides, after the initial recognition of antigen presented on the DCs, the activated CTLs secrete XCL1 that binds with the XCR1 on the DCs to stabilize the DC-CTL contacts. As a result, the upregulated expressions of DC-SIGN and XCR1 on the NZ-maturated mBMDCs might promote and sustain the DC-CTL interactions, which could lengthen the duration of TCR activation and co-stimulation and result in a more efficient activation of CTLs. Furthermore, it is also observed that, compared with the LPS-maturated mBMDCs, the NZ-maturated mBMDCs expressed a higher level of proinflammatory cytokine TNFα and IFNγ and a comparable level of IL 12 after the 20 h maturation period (FIGS. 13A to 13E). The cytokine TNFα can bind to the TNF receptor type II on the CTLs to lower the threshold of TCR activation and serve as the co-stimulatory signals, resulting in the enhanced proliferation and production of IFNγ in CTLs. The IFNγ can act on the CTLs to promote the clonal expansion, production of IFNγ, and differentiation in the CTLs. Therefore, the observation that the NZ-maturated mBMDCs could produce enhanced levels of TNFα and IFNγ after the maturation period, i.e., at the time of interacting with the targeted CTLs, implies their possibly higher capacity to activate the CTLs and their potential in modulating the effector functions and immunological memory of CTLs. Moreover, the capacity of lymphoid homing is another crucial factor for determining the efficacy of DCs in eliciting the anti-tumor CTL response in vivo. CCR7 is a chemokine receptor that recognizes its chemokine ligands, CCL21 and CCL19, expressed by the lymphatic endothelial cells and stromal cells in the lymph nodes, respectively, to guide the entry and promote the homing of DCs to the lymph nodes. Thus, the enhanced level of CCR7 on the NZ-maturated mBMDCs illustrates their higher possibility to migrate to the draining lymph nodes and come into contact with the antigen-specific CTLs, resulting in a promotion of anti-tumor effect induced by the injected DC vaccines. Overall, these findings illustrate that the NZ-maturated mBMDCs acquire a phenotype that potentially benefits the activation of CTLs and the induction of in vivo anti-tumor response.

### Example 4

Mechanisms of the Silica NZ-Promoted Maturation of mBMDCs: Activation of FAK Via the Formation of Curved Cell Adhesions at the DC-NZ Interfaces

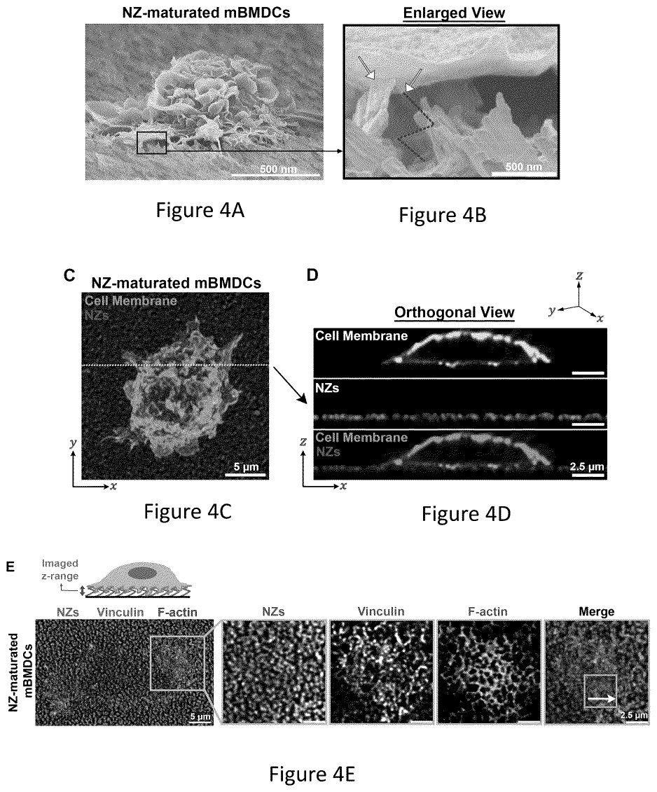

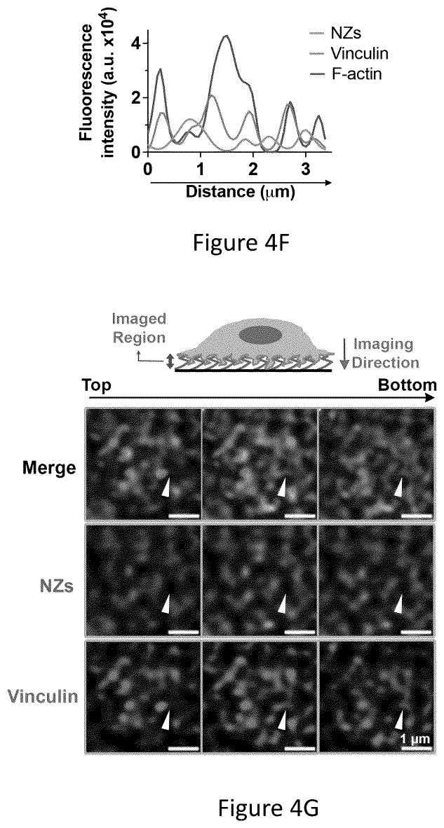

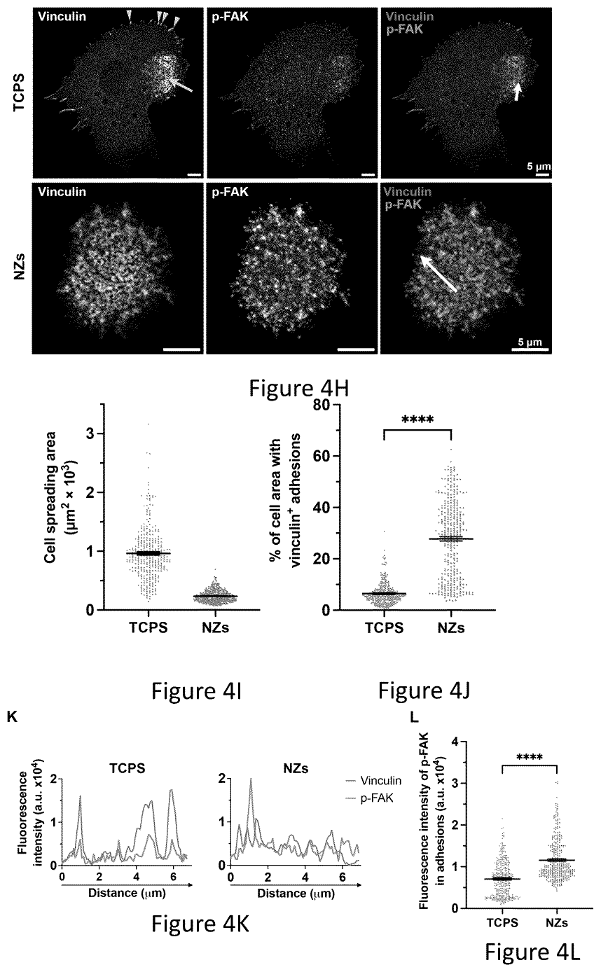

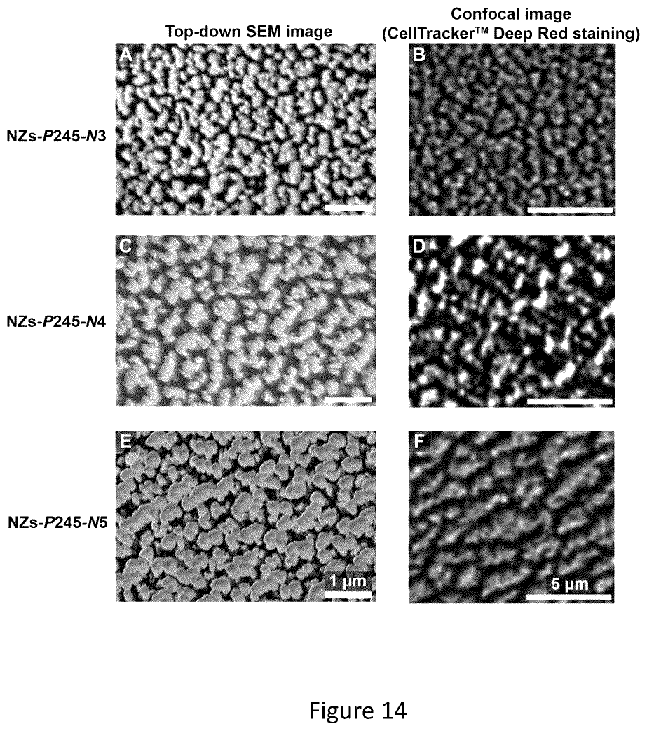

To understand how the silica NZs maturate and enhance the mBMDCs, the inventors first visualized the interaction of silica NZs with mBMDCs using scanning electron microscopy (SEM) and found that silica NZs were in contact with the basal plasma membranes of mBMDCs (FIG. 4A,B). To further visualize how the silica NZs affect the basal plasma membranes of mBMDCs, the inventors stained the plasma membranes of mBMDCs and the silica NZs with CF488A-conjugated wheat germ agglutinin and CellTracker Deep Red dye, respectively. The usage of CellTracker Deep Red dye in the staining of silica NZs was validated by the comparable topographies observed in the top-down SEM images and the confocal images of CellTracker Deep Red dye-stained silica NZs (FIG. 14). The orthogonal views of the stained NZ-maturated mBMDCs showed that the basal plasma membranes of mBMDCs were deformed by the silica NZs (FIGS. 4C, 4D). As cell adhesions are typically formed at the site of contact between the basal plasma membranes and the underlying substrate, the deformation of basal plasma membranes indicates the possible changes in the pattern of cell adhesions in the NZ-maturated mBMDCs. To visualize the cell adhesions in the NZ-maturated mBMDCs, the major cell adhesion molecule, vinculin, lying at the cytoplasmic face of cell adhesions were immune-stained. The actin filament (F-actin) located in the cortical actin under the cell membranes or bundled at the cell adhesions were also stained for visualizing the cell boundaries of mBMDCs. The confocal images of the basal plasma membranes of mBMDCs and the intensity profiles showed that the vinculin+ cell adhesions and F-actin were located at the inter-NZ spaces (FIGS. 4E, 4F), illustrating that the deformed basal plasma membranes of NZ-maturated mBMDCs project into the inter-NZ spaces and the cell adhesions are formed at the DC-NZ interfaces.

Further analysis on the individual slices of confocal z-stack captured at the DC-NZ interfaces revealed the depth of vinculin+ cell adhesions in the NZ-maturated mBMDCs (FIG. 4G), indicating that the NZ-maturated mBMDCs form vertically curved cell adhesions at the lateral surfaces of silica NZs. Previous studies reported that the deformed cell membranes could induce the clustering of membrane curvature-sensitive protein, phosphatidylinositol 4,5-bisphosphate (PIP2), which in turn facilitated the recruitment of vinculin and another mechanosensitive protein FAK to the cell membranes, see G. M. Goni, et al., Proc Natl Acad Sci USA 2014, 111, E3177, and P. M. Thompson, et al., Structure 2017, 25, 264. The binding of FAK to PIP2 can induce conformational change of FAK to expose its Tyr-397 autophosphorylation site, resulting in the activation of FAK.

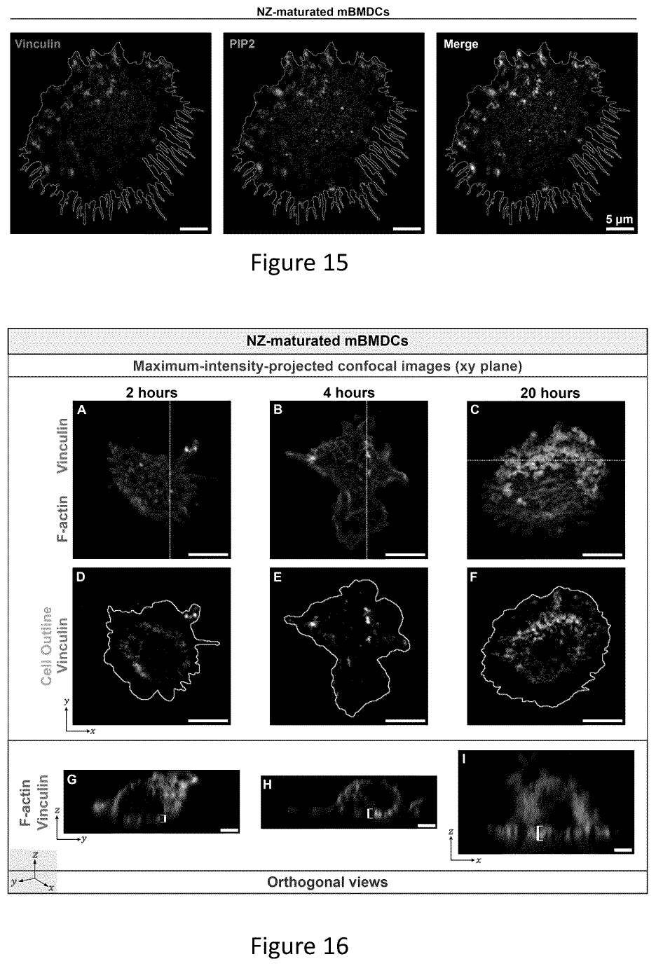

Since the expression of PIP2 was indeed observed in the curved vinculin+ cell adhesions in the NZ-maturated mBMDCs (FIG. 15), the inventors sought to determine whether the activation level of FAK would be upregulated in the cell adhesions of the NZ-maturated mBMDCs. The inventors compared the immunostaining results of vinculin+ cell adhesions and Tyr-397 phosphorylated FAK (p-FAK) in the NZ-maturated mBMDCs with those in the TCPS-attached mBMDCs. The TCPS-attached mBMDCs had larger cell spreading areas and had podosomes and focal adhesions occupying a smaller area of basal plasma membranes as the predominant types of cell adhesions (FIG. 4H-J). In contrast, the NZ-maturated mBMDCs had smaller cell spreading areas, and formed clusters of cell adhesions occupying a larger area of basal plasma membranes (FIG. 4H-J, 16). These observations demonstrate that the silica NZs facilitate the formation of cell adhesions in mBMDCs. Moreover, the confocal images and intensity profiles illustrated that p-FAK was localized in the vinculin+ cell adhesions of both NZ-maturated mBMDCs and TCPS-attached mBMDCs (FIGS. 4H, 4K). Compared with the TCPS-attached mBMDCs, the cell adhesions of NZ-maturated mBMDCs had significantly upregulated levels of pFAK (FIG. 4L). This observation illustrates that the vertically curved cell adhesions formed at the DC-NZ interfaces contain a higher level of active FAK, compared with the podosomes or focal adhesions formed on the flat TCPS. Therefore, these findings indicate that the silica NZs facilitate the mechanoactivation of FAK by inducing the formation of curved cell adhesions at the DC-NZ interfaces.

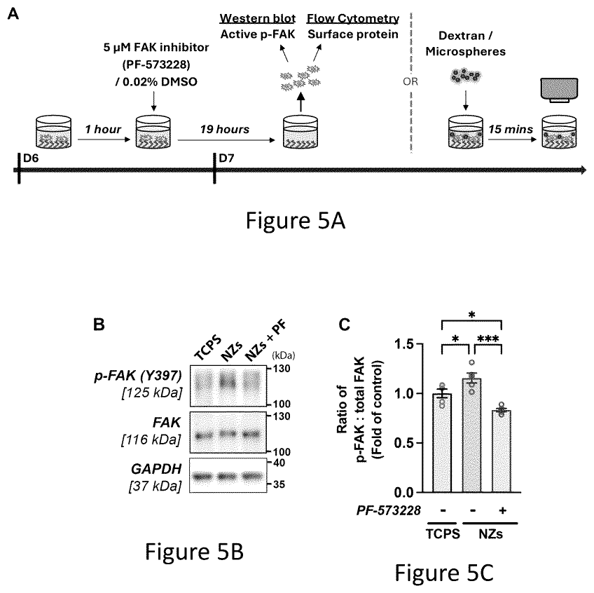

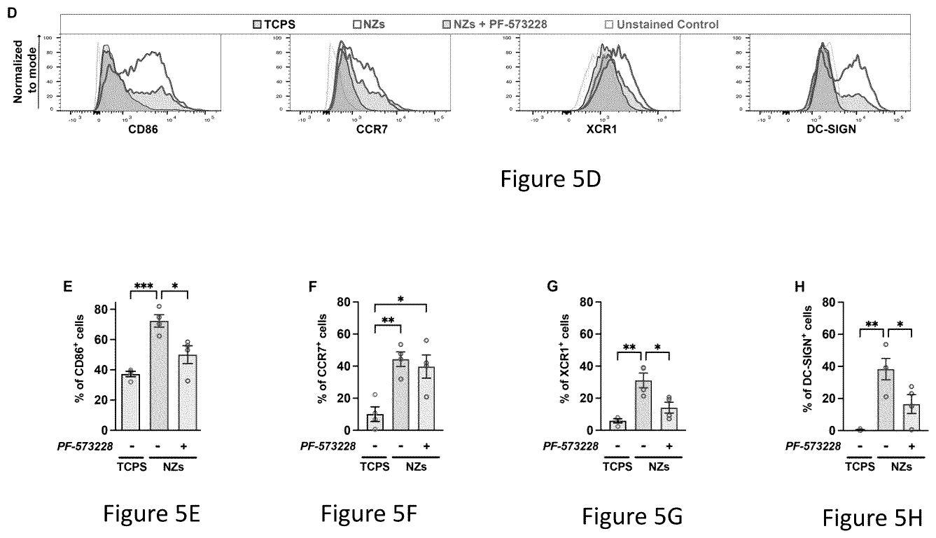

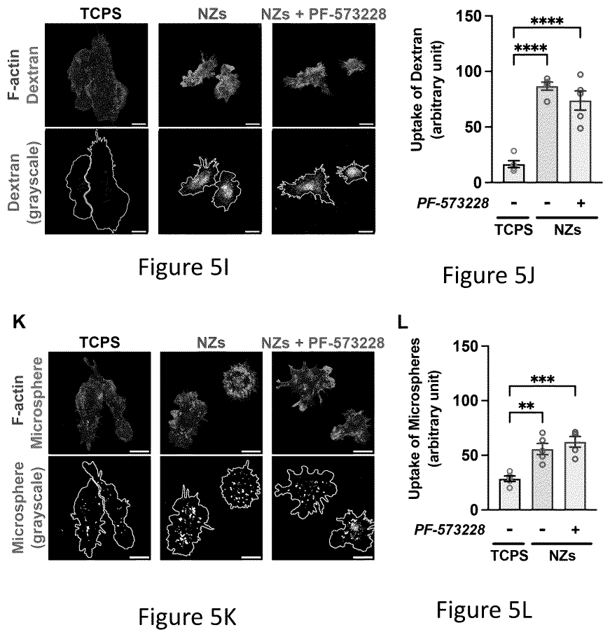

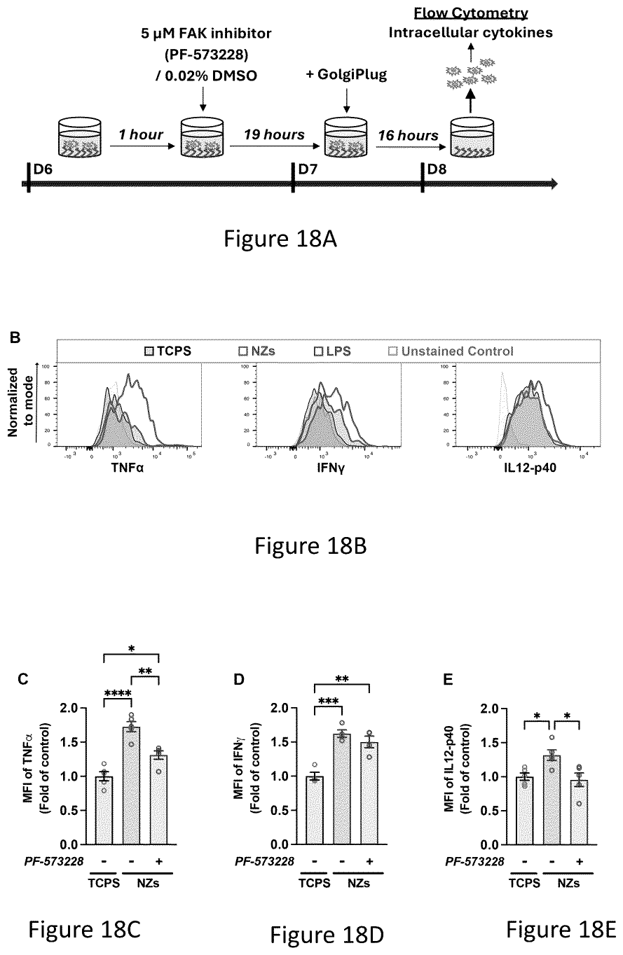

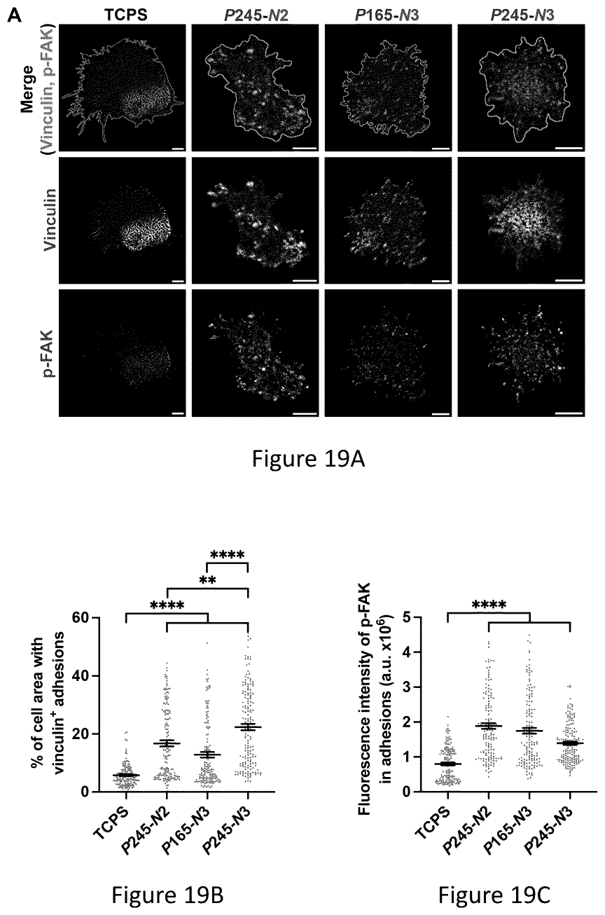

In accordance with the immunostaining results, the upregulation of active p-FAK in the NZ-maturated mBMDCs was confirmed by Western blot assay (FIGS. 5A-C). To further determine the crucial role of FAK in the NZ-mediated maturation of mBMDCs, the activation of FAK in the NZ-maturated mBMDCs was inhibited by the treatment with 5 μm FAK inhibitor, PF-573228 (FIGS. 5A-C). The inhibition of FAK hindered the NZ-induced increase of CD86+, XCR1+, and DC-SIGN⁺ mBMDCs, but not the CCR7⁺ mBMDCs (FIGS. 5D-H, 17A and 17B). However, the enhanced uptake of dextran and microspheres in the NZ-maturated mBMDCs were not affected by the inhibition of FAK (FIGS. 5I-L). Meanwhile, inhibiting the FAK also suppressed the upregulated expressions of TNFα and IL12, but not IFNγ, in the NZ-maturated mBMDCs after the 20 h maturation period (FIGS. 18A to 18E). Moreover, compared with the silica NZs with smaller P or N which led to inferior upregulation of CD86, XCR1, and DC-SIGN in mBMDCs (FIGS. 2D to 2F, 2I, 2J), the optimal silica NZsP245-N3 resulted in the mBMDCs with more abundant cell adhesions that contain high level of active FAK (FIGS. 19A to 19C), demonstrating the possible correlation between the enhanced expression of these surface proteins and the mechanoactivation of FAK in mBMDCs. Therefore, these findings demonstrate that the silica NZs promote the maturation of mBMDCs and upregulate the expressions of CD86, XCR1, and DC-SIGN via FAK activation. FAK is more frequently associated with the motility or formation of cell adhesions in the DCs, while limited studies have reported the roles of FAK in regulating the maturation or protein expression in the DCs.

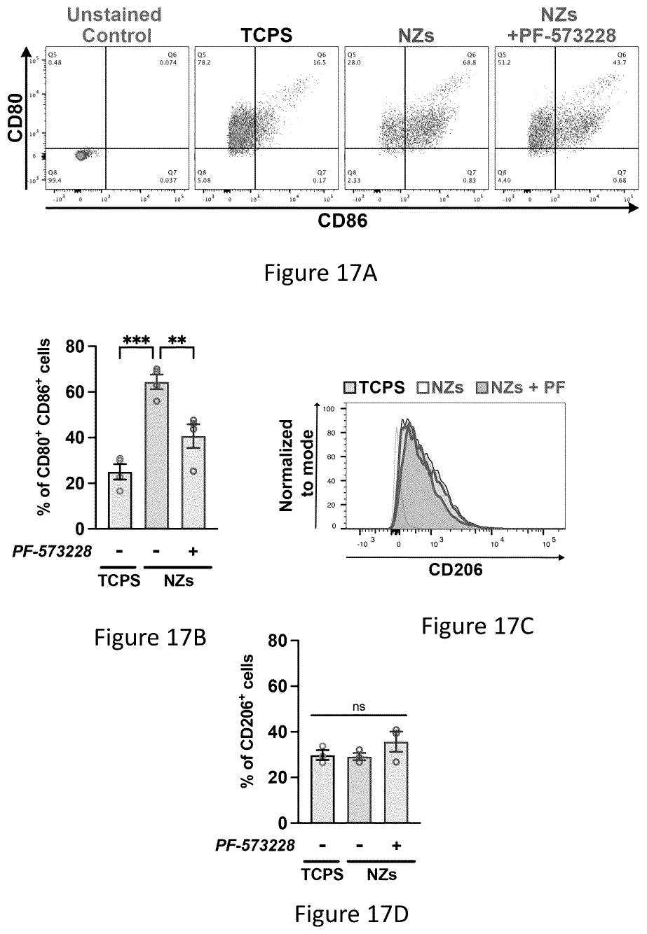

As a non-receptor tyrosine kinase, FAK could possibly phosphorylate multiple downstream targets such as Src kinase and extracellular signal-regulated kinase to initiate the signaling pathway for regulating the protein expression in DCs. Besides, FAK also contains the nuclear localization sequences, allowing it to translocate into the nucleus to regulate the gene transcription or modify the chromatin condensation. It was reported that the nuclear shuttling of FAK was inversely correlated with the kinase activity and membrane recruitment of FAK. Thus, the NZ-induced activation of FAK might regulate the protein expression in mBMDCs by preventing the nuclear-FAK-related suppressions of gene transcription and chromatin modification. On the other hand, the NZ-induced upregulations of CCR7 and IFNγ were found to be independent of the FAK activation. Previous studies have proposed that the nano-patterned substrates could modify the morphologies of cells and nuclei, see M. Ermis, et al., Mater. 2018, 3, 355. The morphological changes could then regulate the nuclear translocation of transcription factors or chromatin modifiers, resulting in changes in gene expression. The distinctive morphological changes, for example, the decrease of cell spreading area, were still observed in the NZ-maturated mBMDCs after the treatment with FAK inhibitor (FIG. 5I,K). This raises a possibility that the silica NZs might also modulate the maturation phenotypes of mBMDCs via the mechanical regulation of transcriptional activity without the involvement of FAK. Moreover, the NZ-induced uptake of dextran and microsphere also did not depend on the FAK activation. The expression level of CD206 was comparable between the TCPS-attached mBMDCs and the NZ-maturated mBMDCs with or without FAK inhibition (FIGS. 17C and 17D). Although the FAK inhibition abolished the NZ-induced upregulation of DC-SIGN, the enhanced internalization of dextran, a ligand of DC-SIGN and CD206, in the NZ-maturated mBMDCs were not notably affected. This implies that the dextran could be internalized into the NZ-maturated mBMDCs via both receptor-mediated endocytosis and non-receptor-mediated pinocytosis or phagocytosis. Previous studies have shown that the nano-topography could enhance the clathrin-mediated endocytosis at the plasma membranes with curvatures induced by the cell-substrate interaction, see W. Zhao, et al., Nat. Nanotechnol. 2017, 12, 750. Thus, the silica NZs might also enhance the endocytic capacity of mBMDCs via the deformation of basal plasma membranes. In short, these findings illustrate that the silica NZs upregulate the expressions of CD86, XCR1, DC-SIGN, TNFα, and IL12 via the FAK activation; and they enhance the expressions of CCR7, IFNγ, and the endocytic capacity possibly by inducing the morphology-regulated gene expressions and the deformation of basal plasma membranes.

### Example 5

In Vitro Effect of the NZ-Maturated mBMDCs on the Activation of Antigen-Specific CTLs

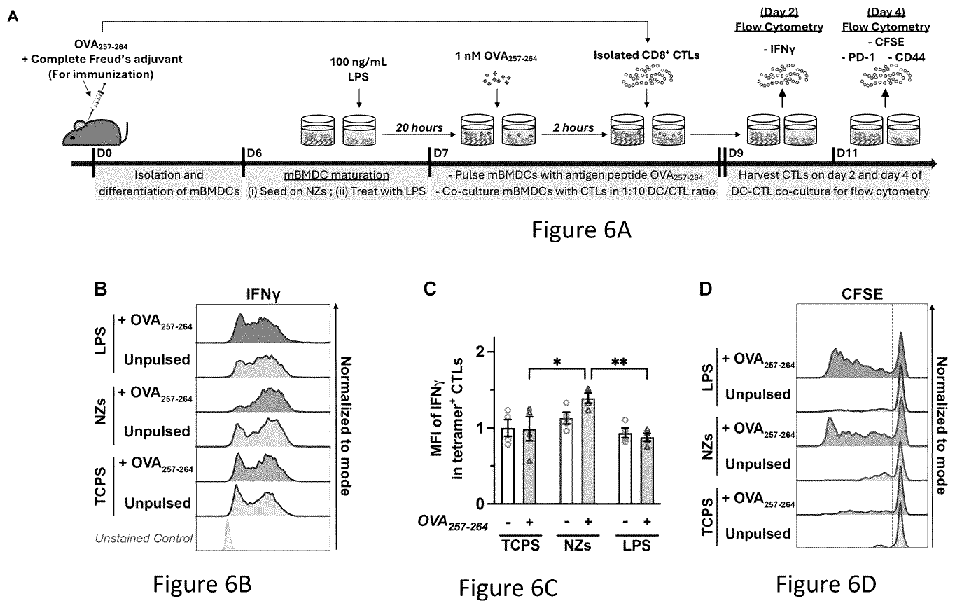

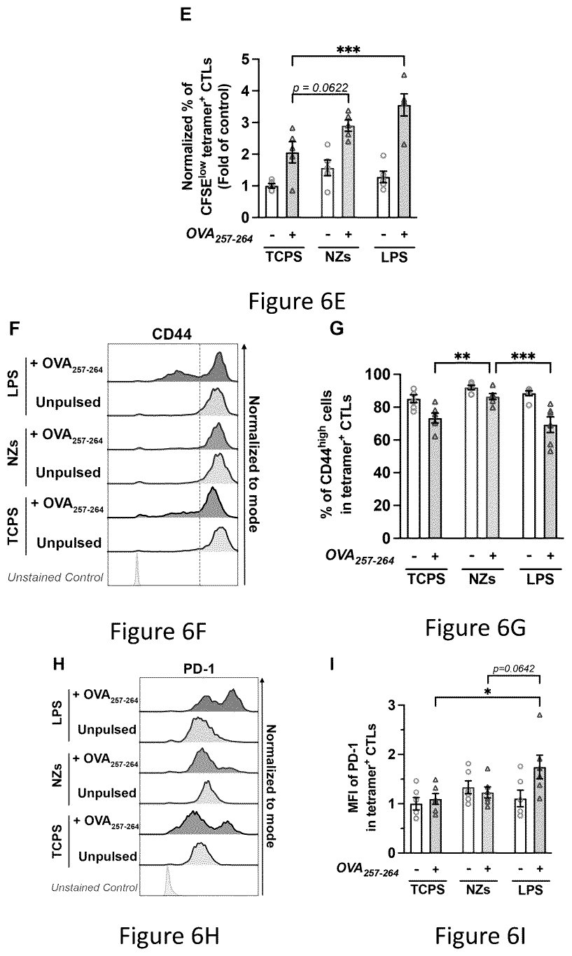

In Example 3, the NZ-maturated mBMDCs were found to have a maturation phenotype different from the LPS-maturated mBMDCs. To examine whether the deviated phenotypes of NZ-maturated mBMDCs would lead to differences in the activation and memory formation of CTLs, the inventors used the MHC-I (H-2k^{b})-restricted OVA₂₅₇₋₂₆₄ peptide as the model antigen to study the different effects of NZ-maturated mBMDCs and LPS-maturated mBMDCs on the priming of antigen-specific CTLs (FIG. 6A).

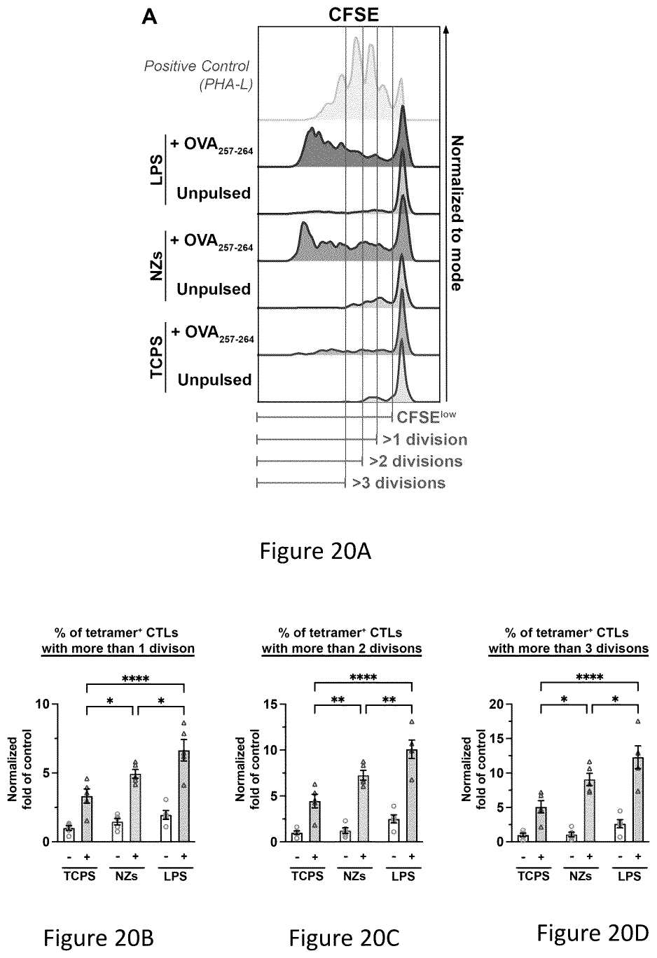

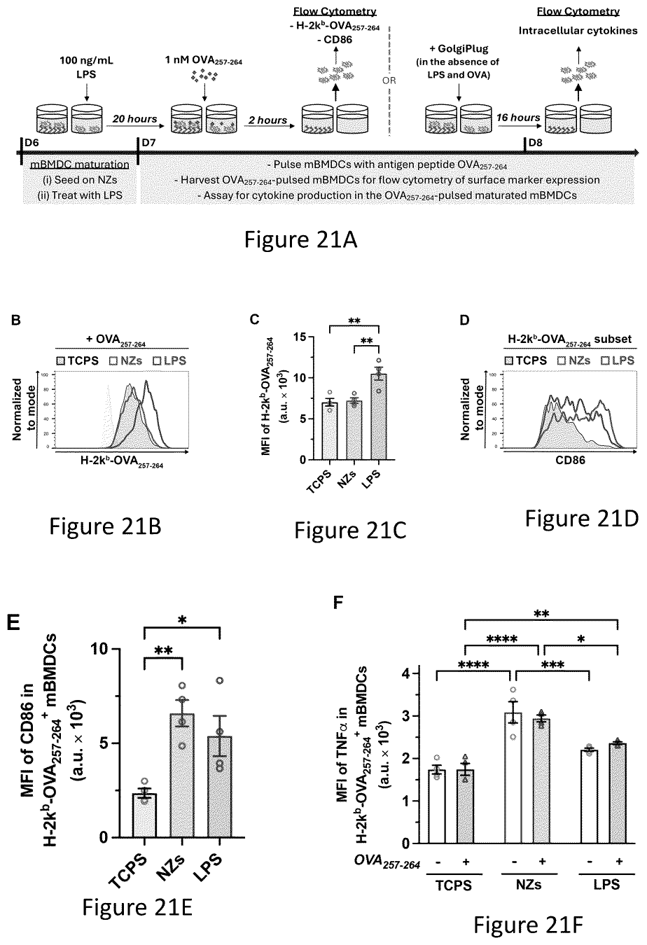

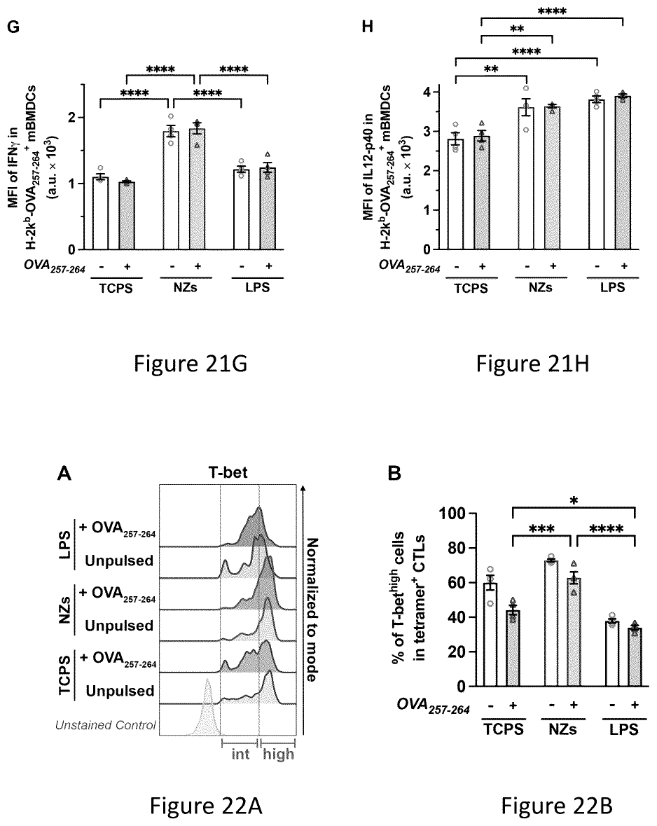

After two days of the DC-CTL co-culture, surprisingly, a higher expression level of IFNγ was detected in the H-2k^{b}-OVA₂₅₇₋₂₆₄-tetramer⁺ antigen-specific CTLs primed by the OVA₂₅₇₋₂₆₄-pulsed NZ-maturated mBMDCs, but not in those primed by the OVA₂₅₇₋₂₆₄-pulsed LPS-maturated mBMDCs (FIG. 6B,C). Meanwhile, according to the results of carboxyfluorescein succinimidyl ester (CFSE)-based CTL proliferation assay obtained after four days of the DC-CTL co-culture, both the OVA₂₅₇₋₂₆₄-pulsed NZ-maturated and LPS-maturated mBMDCs promoted the proliferation of antigen-specific CTLs, wherein the proliferation induced by the OVA₂₅₇₋₂₆₄-pulsed LPS-maturated mBMDCs was more distinctive than those induced by the OVA₂₅₇₋₂₆₄-pulsed NZmaturated mBMDCs (FIGS. 6D and 6E; FIGS. 20A to 20D). Therefore, the CFSE results illustrated that both the NZ-maturated mBMDCs and LPS-maturated mBMDCs pulsed with OVA₂₅₇₋₂₆₄ were able to activate the antigen-specific CTLs, while the NZ-maturated mBMDCs enhanced the production of IFNγ and the LPS-maturated mBMDCs induced a greater proliferation in the antigen-specific CTLs. The LPS-maturated mBMDCs expressed a higher level of MHC-I (FIG. 12E) and OVA₂₅₇₋₂₆₄-bound MHC-I (H-2k^{b}-OVA₂₅₇₋₂₆₄) after the antigen pulsing (FIGS. 21A-21C). Therefore, the higher availability of H-2k^{b}-OVA₂₅₇₋₂₆₄ on the LPS-maturated mBMDCs could increase their chance to ligate with the TCRs on the antigen-specific CTLs, and thus result in a better effect on promoting the proliferation of CTLs.

Although the NZ-maturated mBMDCs only had the baseline expression level of H-2k^{b}-OVA₂₅₇₋₂₆₄ (FIGS. 21B and 21C), their H-2k^{b}-OVA₂₅₇₋₂₆₄⁺ subset had a high expression level of the co-stimulatory molecule CD86 (FIGS. 21D and 21E), which was comparable to those of the LPS-maturated mBMDCs. This ensures the capacity of the OVA₂₅₇₋₂₆₄-pulsed NZ-maturated mBMDCs in inducing the activation and proliferation of the antigen-specific CTLs. Moreover, the production of IFNγ in the activated CTLs is known to be upregulated after the TCR stimulation, while its sustained production is positively regulated by the duration of DC-CTL contact. Thus, no upregulation of IFNγ in the CTLs primed by the OVA₂₅₇₋₂₆₄-pulsed LPS-maturated mBMDCs might be ascribed to that the peak time of IFNγ production had passed. In contrast, in the NZ-maturated mBMDCs, the higher expression of XCR1 can possibly stabilize and prolong the DC-CTL interaction, facilitating the production of IFNγ in the CTLs. Besides, the OVA₂₅₇₋₂₆₄-pulsed NZ-maturated mBMDCs had upregulated expressions of TNFα and IFNγ (FIGS. 21F and 21G) which could directly act on the CTLs to promote the production of IFNγ. Therefore, these properties allow the OVA₂₅₇₋₂₆₄-pulsed NZ-maturated mBMDCs to induce the production of IFNγ and proliferation of the antigen-specific CTLs even without distinctive upregulation of H-2k^{b}-OVA₂₅₇₋₂₆₄.

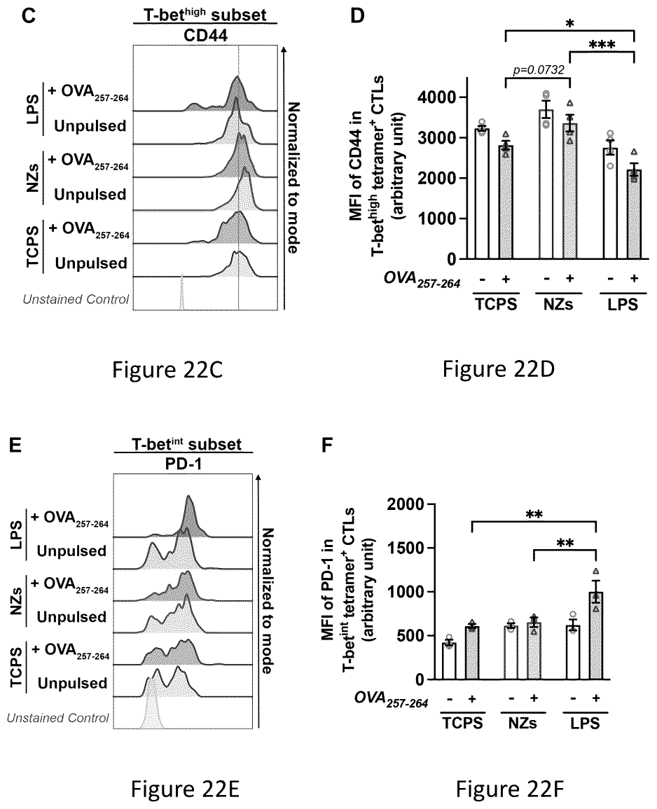

To facilitate the establishment of a long-term anti-tumor immune response, it is desirable for the DC cancer vaccines to promote the differentiation of the antigen-specific CTLs into long-lived memory CTLs with low expression levels of immunoinhibitory molecules. Therefore, the expression levels of memory marker CD44 and immunoinhibitory molecule PD-1 in the antigen-specific CTLs primed by the mBMDCs were also examined in a four-day period. The OVA₂₅₇₋₂₆₄-pulsed NZ-maturated mBMDCs resulted in a higher percentage of the CD44high antigen-specific CTLs without a significant increase of PD-1 (FIG. 6F-I). In contrast, the antigen-specific CTLs primed by the OVA₂₅₇₋₂₆₄-pulsed LPS-maturated mBMDCs had a lower level of CD44 but a significant increase in the expression of PD-1 (FIG. 6F-I). As previously reported, when the CTLs are differentiated into the effector memory cells, the CD44 expression will increase while the PD-1 expression may vary; when the CTLs are differentiated into the terminal effector cells, the PD-1 expression will increase while the CD44 expression will decrease. According to these findings, it is believed that the NZ-maturated mBMDCs and LPS-maturated mBMDCs, with pulsing of OVA₂₅₇₋₂₆₄, tend to induce the differentiation of CTLs into the PD-1^{low}CD44^{high} effector memory CTLs and the PD-1^{high}CD44^{low/int} terminal effector CTLs, respectively. The expressions of CD44 and PD-1 in CTLs are oppositely regulated by a master transcription factor, T-box transcription factor TBX21 (T-bet), which promotes the expression of CD44 but suppresses that of PD-1. The OVA₂₅₇₋₂₆₄-pulsed NZ-maturated mBMDCs resulted in a higher proportion of T-bet^{high} antigen-specific CTLs which had upregulated levels of CD44 (FIGS. 22A to 22D). In contrast, the OVA₂₅₇₋₂₆₄-pulsed LPS-maturated BMDCs gave rise to antigen-specific CTLs with intermediate levels of T-bet and higher levels of PD-1 (FIGS. 22A, 22B, 22E and 22F). These results indicate that the deviated expressions of CD44 and PD-1 in the primed CTLs are regulated by the T-bet. The cytokine IFNγ is one of the major cytokines to amplify the expression of T-bet in the CTLs. The results showed that the OVA₂₅₇₋₂₆₄-pulsed NZ-maturated mBMDCs had higher expression levels of TNFα and IFNγ (FIGS. 21F to 21G), which could possibly act on the CTLs in a paracrine manner to increase the production of IFNγ in the CTLs.

The CTLs primed by the OVA₂₅₇₋₂₆₄-pulsed NZ-maturated mBMDCs indeed had a higher level of IFNγ (FIG. 6B,C), which could possibly function in both autocrine and paracrine manners to increase the expression of T-bet in the CTLs. Therefore, it is demonstrated from the findings that the antigen-pulsed NZ-maturated mBMDCs are capable of activating the antigen-specific CTLs and promoting the differentiation of CTLs into PD-1^{low}CD44^{high} effector memory CTLs possibly via the TNFα-IFNγ-T-bet pathway.

### Example 6

In Vivo Effect of the NZ-Maturated mBMDCs on Suppression of Tumor Growth

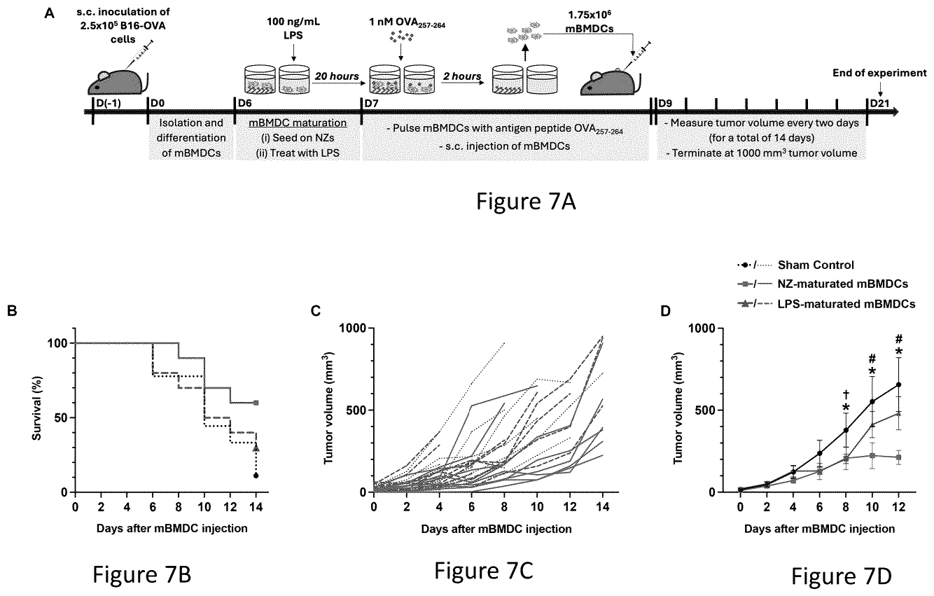

To understand whether the deviated phenotypes of the NZ-maturated mBMDCs would benefit the elimination of cancer cells in vivo, the inventors compared the effects of OVA₂₅₇₋₂₆₄-pulsed NZ-maturated mBMDCs and OVA₂₅₇₋₂₆₄-pulsed LPS-maturated mBMDCs on suppressing the tumor growth in the mice bearing the melanoma B16-OVA tumors (FIG. 7A). Compared with the sham control group which was injected with the vehicle (HBSS) without mBMDCs, a single injection of the OVA₂₅₇₋₂₆₄-pulsed NZ-maturated mBMDCs enhanced the survival rate of tumor-bearing mice over the course of 14 days (FIG. 7B), which was not observed for the OVA₂₅₇₋₂₆₄-pulsed LPS-maturated mBMDCs. Compared with the sham control group, on day 8 after the mBMDC injection, both the OVA₂₅₇₋₂₆₄-pulsed NZ-maturated and LPS-maturated mBMDCs made the tumor volume decrease. However, from day 10 after the mBMDC injection, only the OVA₂₅₇₋₂₆₄-pulsed NZ-maturated mBMDCs significantly suppressed the growth of tumor (FIGS. 7C, 7D). These findings indicate that the anti-tumor effects induced by the NZ-maturated mBMDCs are more long-lasting than those induced by the LPS-maturated mBMDCs. These deviated anti-tumor effects are likely due to that the OVA₂₅₇₋₂₆₄-pulsed NZ-maturated mBMDCs promoted the generation of relatively long-lived PD-1^{low}CD44^{high} memory effector CTLs in mice, while the OVA₂₅₇₋₂₆₄-pulsed LPS-maturated mBMDCs led to the short-lived PD-1^{high}CD44^{low} terminal effector CTLs. Moreover, the PD-1^{low}CD44^{high} CTLs were reported to be rescued from apoptosis and to restore their expansion and effector functions via the application of immune checkpoint inhibitors, raising the potential to further improve the anti-tumor effect via a combinational treatment of NZ-maturated DCs and immune checkpoint inhibitors. In short, these findings verify that the NZ-maturated mBMDCs are advantageous over the LPS-maturated mBMDCs in the suppression of tumor growth in vivo.

### Example 7

### Silica NZ-Promoted Maturation of Human Monocyte-Derived Dendritic Cells

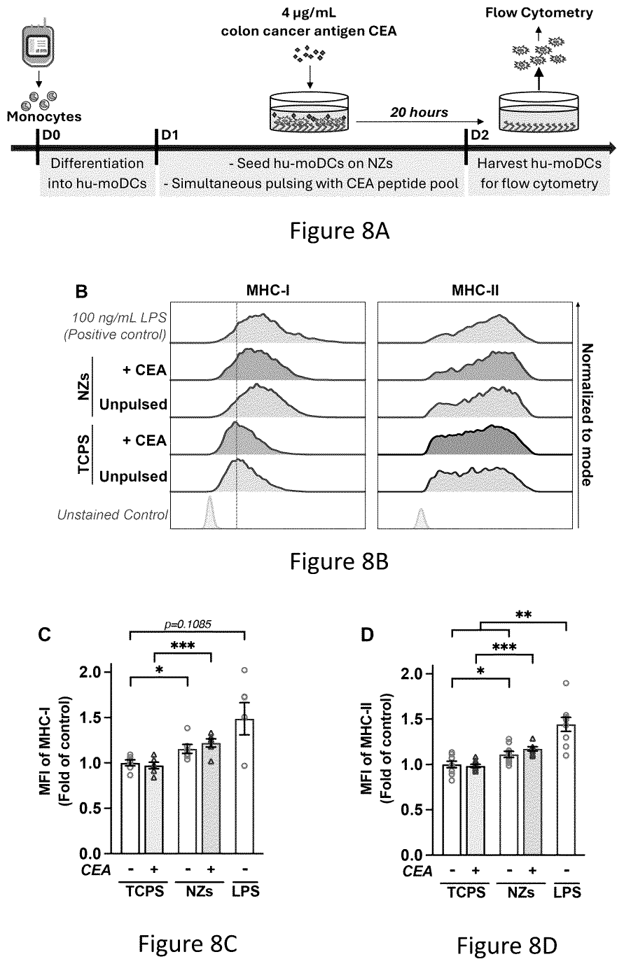

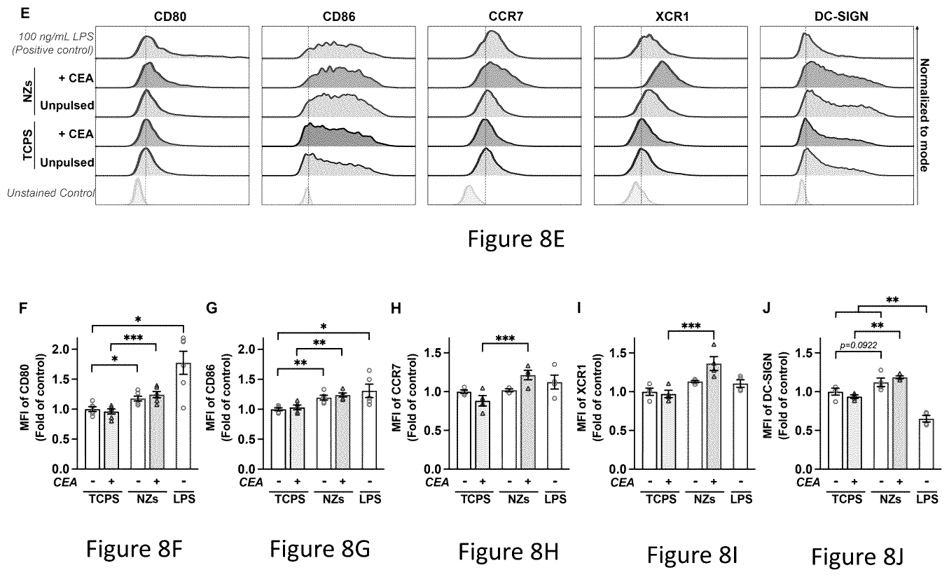

The effects of silica NZs on the maturation of hu-moCs differentiated via a rapid and serum-free protocol was examined. The detailed protocol can be found in F. Tanaka, et al., Int J Oncol 2006, 29, 1263. The peptide pool of colon cancer-related antigen, carcinoembryonic antigen (CEA), was used as the model tumor-associated antigen (FIG. 8A).

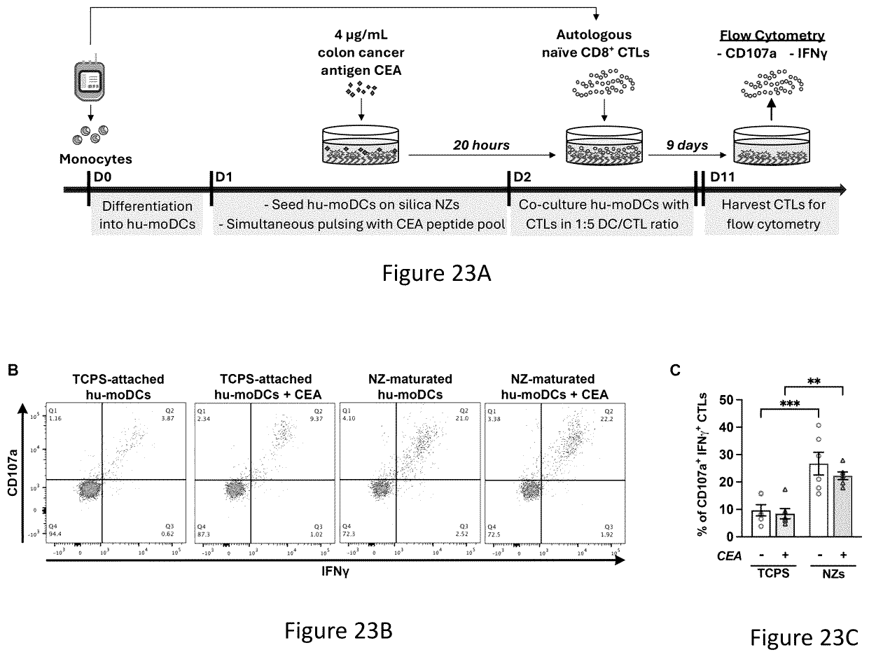

Unlike the results on the mBMDCs, the silica NZs significantly increased the expression levels of MHC-I and MHC-II in the hu-moDCs with or without pulsing with CEA (FIGS. 8B-D), indicating that the NZ-induced expressions of MHC molecules are obscured in the mBMDCs or in the serum-supplemented culture system. Consistently with the observations on the mBMDCs, the silica NZs also upregulated the expressions of the co-stimulatory molecule CD80 and CD86 in the hu-moDCs with or without pulsing with CEA (FIGS. 8E-G). However, the distinctively enhanced expressions of CCR7, XCR1, and DC-SIGN were only observed in the NZ-maturated hu-moDCs pulsed with CEA, but not in those without antigen pulsing (FIGS. 8E, 8H-8J). The pulsing with CEA did not cause significant changes in the TCPS-attached hu-moDCs (FIGS. 8H-8J), illustrating that CEA might initiate another signaling to amplify the NZ-mediated upregulation of CCR7, XCR1, and DC-SIGN. It has been reported that the glycosylated CEA is a ligand for DC-SIGN, K. P. van Gisbergen, et al., Cancer Res. 2005, 65, 5935, and the activation of DC-SIGN can modulate the maturation of DCs. The oligopeptides of CEA could possibly be glycosylated by the reducing sugars such as glucose in the culture medium via the Maillard reaction. The silica NZs had slightly upregulated the expression of DC-SIGN in the unpulsed hu-moDCs (p=0.0922, FIGS. 8E, 8J). Hence, it is possible that the glycosylated oligopeptides of CEA ligate with the DC-SIGN on the NZ-maturated humoDCs to augment the expressions of CCR7, XCR1, and DCSIGN. Moreover, enhanced degranulation and production of IFNγ were observed in the CTLs co-cultured with the NZ-maturated hu-moDCs (FIGS. 23A to 23C), demonstrating the capacity of NZ-maturated hu-moDCs to activate the CTLs. Overall, these findings demonstrate the feasible application of silica NZs to maturate the anti-tumor hu-moDCs in vitro.

### CONCLUSION

mBMDCs were used as a cell model to manifest the capacity of silica nanomatrices in promoting in vitro maturation of DCs. The outcome of silica nanomatrix-mediated maturation can be modulated via the engineering of nanostructures in terms of shapes, P, and N. The optimal silica nanomatrices, NZs-P245-N3, generate a maturation phenotype consisting of upregulated expressions of CD86, CCR7, XCR1, and DC-SIGN, and elevate the endocytic capacity of DCs. Compared with the conventionally LPS-maturated mBMDCs, the silica NZs-P245-N3 maturate the mBMDCs to acquire higher expression levels of CCR7, XCR1, and DC-SIGN, as well as to retain the enhanced endocytic capacity after the maturation period. The NZs-P245-N3-induced upregulations of CD86, XCR1, and DC-SIGN are mediated by FAK that is mechanically activated at the curved cell adhesions formed in the deformed plasma membranes at the DC-NZ interfaces. The distinctive maturation phenotypes of NZs-P245-N3-maturated mBMDCs enable the activation of antigen-specific CTLs into the PD-1^{low}CD44^{high} effector memory CTLs in vitro and a relatively prolonged suppression of tumor growth in vivo. Moreover, the NZs-P245-N3-induced surface phenotypes of 5 maturated DCs are reproducible in the hu-moDCs. Whereas the NZs-P245-N3-induced increase of MHC molecules is observed in the hu-moDCs maintained in the serum-free condition but concealed in the mBMDCs cultured in the serum-supplemented system. The results herein demonstrate the superior effects of silica NZs on in vitro maturation of anti-tumor DCs in both murine and human cells, implying their potential to be developed into a new strategy of cellular immunotherapy for cancer treatment.

1. A method of producing matured dendritic cells for immunotherapy, comprising:
a) providing a biocompatible nanomatrix;
b) seeding immature dendritic cells onto the biocompatible nanomatrix; and
c) culturing the dendritic cells for a period sufficient to induce maturation via mechanical stimuli provided by the biocompatible nanomatrix.

2. The method of claim 1, wherein the biocompatible nanomatrix comprises a substrate, and a plurality of nanostructures protruding from the substrate.

3. The method of claim 2, wherein the nanostructures are composed of silica.

4. The method of claim 2, wherein the nanostructures are configured in a zigzag geometry.

5. The method of claim 4, wherein the zigzag geometry is configured to induce mechanical maturation of dendritic cells via activation of focal adhesion kinase.

6. The method of claim 2, wherein each of the nanostructures comprises between two and five pitches.

7. The method of claim 6, wherein each of the nanostructures comprises 3 pitches.

8. The method of claim 6, wherein each of the pitches has a height of approximately 100 nm to 500 nm.

9. The method of claim 2, wherein the nanostructures are fabricated via glancing angle deposition.

10. The method of claim 1, wherein the immature dendritic cells are bone marrow-derived dendritic cells, monocyte-derived dendritic cells, or CD34⁺ hematopoietic stem cells.

11. The method of claim 1, wherein the period sufficient to induce maturation is approximately 20 hours.

12. The method of claim 1, wherein the maturation of step c) is performed in the absence of exogenous chemical maturation agents or cytokines.

13. The method of claim 1, wherein the matured dendritic cells are configured to activate antigen-specific cytotoxic T-lymphocytes.

14. The method of claim 13, wherein the activated antigen-specific cytotoxic T-lymphocytes exhibit a PD-1^{low}CD44^{high} memory phenotype.

15. The method of claim 1, further comprising pulsing the dendritic cells with at least one tumor-associated antigen.

16. The method of claim 1, wherein the matured dendritic cells exhibit upregulated expression of a co-stimulatory and immunoregulatory molecule selected from the group consisting of CD86, DC-SIGN, XCR1, CCR7, IL-12, TNFα, and a combination thereof.

17. Matured dendritic cells prepared according to the method of claim 1.

18. A pharmaceutical composition comprising an effective amount of the matured dendritic cells of claim 17.

19. The pharmaceutical composition of claim 18, wherein the matured dendritic cells present at least one tumor-associated antigen on the MHC-I complexes.

20. The pharmaceutical composition of claim 18, wherein the matured dendritic cells exhibit upregulated expression of a co-stimulatory and immunoregulatory molecule selected from the group consisting of CD86, DC-SIGN, XCR1, CCR7, IL-12, TNFα, and a combination thereof.
